(19) United States
(12) Patent Application Publication
(10) Pub. No.: US 2026/0215901 A1
(43) Pub. Date: Jul. 30, 2026

(54) POULTRY PROCESSING SYSTEMS AND METHODS

(71) Applicant: NOVA-TECH ENGINEERING, LLC, Willmar, MN (US)

(72) Inventors: Eric A. Hillesheim, Spicer, MN (US); Gary Johnson, Willmar, MN (US); Austin Mueller, Atwater, MN (US); William Van Dijk, Willmar, MN (US); Brandon Masters, Belgrade, MN (US); Anthony Mickle, Atwater, MN (US); Marcos Rodriguez, Litchfield, MN (US); Andrew Hokom, New London, MN (US); Matthew Biel, Maple Grove, MN (US)

(21) Appl. No.: 19/148,492

(22) PCT Filed: Jan. 16, 2024
(86) PCT No.: PCT/US2024/011565

§ 371 Date: Jul. 16, 2025

Related U.S. Application Data
(60) Provisional application No. 63/439,478, filed on Jan. 17, 2023.

(51) Int. Cl. A61D 1/02; A61D 3/00; A61D 7/00

(52) U.S. Cl. CPC A61D 1/025; A61D 3/00; A61D 7/00; A61D 2003/003

(57) ABSTRACT

Poultry processing systems, piston pump assemblies, and modular pump bases along with related methods are described herein. The poultry processing systems may include injection stations that allow for adjustment of an approach angle relative to a bird in a bird support aligned with the injection stations. The poultry processing systems may include piston pump assemblies using dosage pucks to provide accurate control over the volume of each bolus of liquid delivered with each activation of the piston pump assemblies. The poultry processing systems may include pumping bases and pump identification systems to allow for easy and quick changeover of pumps associated with the poultry processing systems.

### RELATED APPLICATION

This application claims the benefit under 35 U.S.C. § 119 of U.S. Provisional Application Ser. No. 63/439,478 filed 17 Jan. 2023 and titled POULTRY

PROCESSING SYSTEMS AND METHODS, which is incorporated herein by reference in its entirety.

### FIELD

Poultry processing systems, piston pump assemblies, and modular pump bases along with related methods are described herein.

### BACKGROUND

The processing of poultry may include activities such as sexing to determine gender, inoculating or otherwise medicating the birds, feeding the birds, weighing the birds, treating the beaks and/or claws of the birds (to, e.g., retard their growth), etc. In some conventional systems, birds are handled manually, i.e., individuals must physically hold the bird to perform the injection process.

When injecting poultry to deliver an inoculant such as, e.g., one or more medications or other therapeutic substance, vitamins, or any other substance that should or could be advantageously delivered subcutaneously, the injection process may be complicated by the smaller size of the birds and their movement. Further, in some instances, it may be desirable to deliver the same or different injectable materials to a bird.

Injection of liquids through needles having lumens is typically limited to the delivery of one liquid through the needle due to limitations in the liquid delivery equipment. Systems developed for the delivery of two or more different liquids through needles typically employ different needles for each liquid to be delivered. Some examples of such systems may be described in, for example, International Publication WO 2018/204572 (Eid et al.) and U.S. Pat. No. 4,758,227 (Lancaster, Jr. et al.).

### SUMMARY

Poultry processing systems, piston pump assemblies, and modular pump bases along with related methods are described herein.

In one or more embodiments, the poultry processing systems described herein may include injection stations that define an approach angle relative to a bird in a bird support aligned with the injection stations. The approach angle is the angle at which an injection axis along which an injection needle moves relative to a platform axis about which bird supports rotate into and out of alignment with the injection stations of a poultry processing system as described herein.

Adjustment of the approach angle may provide a variety of benefits. For example, adjustment of the approach angle may provide for more consistent delivery of liquids through an injection needle based on bird anatomy. In particular, variations in bird anatomy varies may result in inefficient or ineffective subcutaneous delivery of fluids through an injection needle. For example, some birds have, e.g., flatter backs, larger heads, and/or other anatomical differences that may make adjustment of the approach angle advantageous when setting up a poultry processing system as described herein. Anatomical differences may, of course, be found between different species of birds (e.g., chickens, ducks, turkeys, etc.) but anatomical differences that can affect delivery of injectable materials using the poultry processing systems described herein can be found within the same species or even between flocks of birds.

One or more embodiments of the poultry processing systems described herein incorporate features that allow for adjustment of the approach angle of an injection needle as described herein without changing the position of the delivery site of the injection needle. During development of the injection stations and poultry processing systems described herein, it has been found that consistent positioning of the delivery site of the injection needle between different approach angles is beneficial because it avoids the need to change any positioning of the bird supports relative to the remainder of the poultry processing system such as, e.g., the platform axis, etc.

In one or more embodiments of the poultry processing systems described herein the approach angle of an injection needle of an injection station without changing the position of the delivery site of the injection needle can be accomplished using one or more shims positioned between a base of an injection station and the system platform.

Using shims positioned between the injection stations and the system platform to adjust the approach angles as described herein provides an additional advantage because the injection stations themselves may be uniform, i.e., require no additional adjustments, which can simplify manufacturing of the injection stations, set up of poultry processing systems using the injection stations, replacement of components of the injection stations, etc. Because shims have a fixed or set size and shape, the use of shims to adjust approach angles eliminates the need to make adjustments that might vary between injection stations due to, e.g., human error, slippage (due, e.g., to improperly tightened connectors), etc.

The use of shims to adjust approach angles is particularly useful in poultry processing systems in which bird supports have a fixed orientation relative to, e.g., the platform axes or other stationary features of the poultry processing systems described herein. Because the bird supports present birds retained in the bird supports to the processing stations of the poultry processing systems at a fixed orientation and position relative to stationary features such as, e.g., the injection stations, the approach angles defined by the injection axes of the injection stations are uniform when the injection stations are mounted on the system platforms using either no shims or the same shims.

In one or more embodiments of the piston pump assemblies (that may be used in one or more embodiments of the poultry processing systems) described herein, accurate control over the volume of each bolus of liquid delivered with each activation of the piston pump assemblies may be important to ensure efficacy and safety. Control over accurate bolus delivery can be achieved by controlling the travel of a piston, but such control is often difficult to obtain, particularly in situations in which multiple different boluses must be delivered using the same piston pump.

The use of dosage pucks to control the travel of a piston in a piston pump in one or more embodiments of the piston pump assemblies described herein addresses the need for accuracy by separating piston travel control from the piston pump itself. In one or more embodiments, the dosage pucks have selected heights that correspond to a selected travel distance that, in turn, corresponds to a selected volume within the piston chamber such that the stroke distance of the piston within the piston pump can be controlled precisely using dosage pucks having known heights.

The pumps used to deliver liquids in the modular injection systems described herein may be chosen for their ability to accurately deliver selected boluses of liquids to an injection needle. In one or more embodiments, peristaltic pump assemblies may be used to deliver one liquid having lower viscosity and/or lower density to an injection needle assembly while a piston pump assembly may be used to deliver a different liquid having a higher viscosity and/or higher density to the needle assembly. The viscosity and/or density differences may, in one or more embodiments, be the result of different carrier liquids (e.g., oils versus water-based carriers) used in the different liquids.

Because the liquids delivered in the poultry processing systems described herein may present different pumping challenges, it may be desirable to allow for easy and quick changeover of pumps associated with the poultry processing systems. In such situations, it may be desirable to have a poultry processing system capable of identifying the types of pumps being used and adjust the control system for accurate operation of the different types of pumps. In one or more embodiments of the poultry processing systems described herein pump identification systems are incorporated into the poultry processing system to allow for that pump identification and modification of the control systems operating those pumps.

In a first aspect, one or more embodiments of a poultry processing system as described herein include: a system platform and a support ring mounted on a base, wherein the system platform comprises an injection sector comprising a plurality of mounting sites, wherein one or both of the system platform and the support ring are configured to rotate relative to each other about a platform axis extending through the system platform, the support ring, and the base; a plurality of bird supports mounted on a support ring outside of a perimeter of the system platform, wherein each bird support of the plurality of bird supports is in a selected orientation relative to the platform axis at each mounting site of the plurality of mounting sites, wherein rotation of one or both of the system platform and the support ring about the platform axis moves the plurality of bird supports into and out of alignment with the plurality of mounting sites, and wherein each bird support of the plurality of bird supports is configured to retain a bird in a selected orientation relative to the platform axis; an injection station attached to the system platform at a selected mounting site of the plurality of mounting sites. The injection station comprises: a needle assembly comprising an injection needle, a needle guide, and a needle actuator operably connected to the injection needle, wherein the needle actuator is configured to move the injection needle between an injection position and a retracted position along an injection axis, wherein a distal end of the injection needle extends out of a delivery site of the needle guide along the injection axis when the injection needle is in the injection position, and an injection carriage operably connected to a carriage actuator, wherein the needle assembly, the needle guide, and the needle actuator are mounted on the injection carriage, wherein the carriage actuator is configured to rotate the injection carriage about a carriage axis between a standby position and an actuation position, and wherein the injection axis defines a base approach angle relative to the platform axis when the injection carriage is in the actuation position such that the injection needle approaches a bird support positioned at the selected mounting site at the base approach angle. The poultry processing system also includes a shim configured to be positioned between the base of the injection station and the selected mounting site, wherein, when positioned between the base of the injection station and the selected mounting site, the shim positions the base above the selected mounting site and rotates the injection axis of the injection station to an adjusted approach angle relative to the platform axis such that the injection needle approaches a bird support positioned at the selected mounting site at the adjusted approach angle.

In one or more embodiments of the poultry processing systems described herein, the injection axis of the injection station rotates about the delivery site of the needle guide when moving between the base approach angle and the adjusted approach angle such that the delivery site of the needle guide is positioned at the same location relative to the platform axis when the injection axis is in the base approach angle and when the injection axis is in the adjusted approach angle. In one or more embodiments, the shim rotates the injection axis about the delivery site 2 degrees or more, 5 degrees or more, 10 degrees or more, 15 degrees or more, or 20 degrees or more, and, optionally, 30 degrees or less, 25 degrees lor less, 20 degrees or less, 15 degrees or less, or 10 degrees or less.

In one or more embodiments of the poultry processing systems described herein, the injection axis extends through the carriage axis.

In one or more embodiments of the poultry processing systems described herein, the injection station attached to the system platform at the selected mounting site comprises a first injection station of a plurality of injection station attached to the system platform, wherein the plurality of poultry processing system comprises a second injection station attached to the system platform at a second selected mounting site of the plurality of mounting sites, and wherein the second injection station comprises: a second needle assembly comprising a second injection needle, a second needle guide, and a second needle actuator operably connected to the second injection needle, wherein the second needle actuator is configured to move the second injection needle between an injection position and a retracted position along a second injection axis, wherein a distal end of the second injection needle extends out of a second delivery site of the second needle guide along the second injection axis when the second injection needle is in the second injection position, and a second injection carriage operably connected to a second carriage actuator, wherein the second needle assembly, the second needle guide, and the second needle actuator are mounted on the second injection carriage, wherein the second carriage actuator is configured to rotate the second injection carriage about a second carriage axis between a standby position and an actuation position, and wherein the second injection axis defines a second base approach angle relative to the platform axis when the second injection carriage is in the actuation position such that the second injection needle approaches a bird support positioned at the second selected mounting site at the second base approach angle, wherein the base approach angle of the first injection station and the second base approach angle are the same. In one or more embodiments, the poultry processing system comprises a second shim configured to be positioned between the base of the second injection station and the second selected mounting site, wherein, when positioned between the base of the second injection station and the second selected mounting site, the second shim positions the base of the second injection station above the second selected mounting site and rotates the second injection axis of the second injection station to a second adjusted approach angle relative to the platform axis such that the second injection needle approaches a bird support positioned at the second selected mounting site at the second adjusted approach angle. In one or more embodiments, the second injection axis of the second injection station rotates about the second delivery site of the second needle guide when moving between the second base approach angle and the second adjusted approach angle such that the second delivery site of the second needle guide is positioned at the same location relative to the platform axis when the second injection axis is in the second base approach angle and when the second injection axis is in the second adjusted approach angle.

In one or more embodiments of the poultry processing systems described herein, each shim of the plurality of shims positions the base and the injection axis of each injection station of the plurality of injection station at the same selected adjusted angle relative to the platform axis.

In one or more embodiments of the poultry processing systems described herein, the injection station comprises a carriage damper operably connected to the injection carriage, the carriage damper configured to limit rotational velocity of the injection carriage during rotation of the injection carriage from the standby position to the actuation position. In one or more embodiments, the carriage damper comprises a one-way damper such that the carriage damper is configured to limit rotational velocity of the injection carriage only during rotation of the injection carriage from the standby position to the actuation position. In one or more embodiments, the carriage damper comprises a rotary damper. In one or more embodiments, the carriage damper comprises a passive damper.

In one or more embodiments of the poultry processing systems described herein, the system comprises a piston pump assembly in fluid communication with the injection needle of the injection station, wherein the piston pump assembly comprises: a piston pump comprising: a housing defining a piston chamber, a chamber inlet in fluid communication with the piston chamber, a chamber outlet in fluid communication with the piston chamber, a flow control apparatus configured to prevent fluid from leaving the piston chamber through the chamber inlet, a piston comprising a forward end located in the piston chamber and a trailing end located outside of the piston chamber, the piston configured for movement along a piston axis within the piston chamber, wherein movement of the forward end of the piston along the piston axis within the piston chamber towards the chamber outlet is configured to force fluid in the piston chamber out of the piston chamber through the chamber outlet, and biasing apparatus configured to move the piston to a rest position, wherein the biasing apparatus is configured to resist movement of the piston towards the chamber outlet; a pump actuator comprising a piston driver, wherein the pump actuator is configured to move the piston driver towards the chamber outlet to an advanced position, wherein the piston driver is proximate the trailing end of the piston when the piston driver is in the advanced position and the piston is in the rest position; and a dosage puck configured for placement between the trailing end of the piston and the piston driver, wherein the dosage puck comprises a selected height that corresponds to a selected volume within the piston chamber between the forward end of the piston and the chamber outlet such that movement of the piston towards the chamber outlet over a stroke distance equal to the selected height of the dosage puck moves the selected volume of fluid out of the piston chamber through the chamber outlet.

In one or more embodiments of the poultry processing systems including a piston pump assembly as described herein, the piston driver is spaced away from the advanced position when the dosage puck is located between the piston driver and the trailing end of the piston, and wherein the assembly comprises a controller operably connected to the piston actuator, wherein the controller is configured to: optionally operate the piston actuator to bias the piston driver towards the advanced position with a base force such that when the dosage puck is located between the piston driver and the trailing end of the piston, the piston driver exerts a driving force equal to or less than the base force on the trailing end of the piston; and selectively operate the piston actuator to bias the piston driver towards the advanced position with a pumping force greater than the base force, wherein the pumping force is large enough such that the piston driver moves towards the advanced position and the piston is moved towards the chamber outlet over the stroke distance when the dosage puck is located between the piston driver and the trailing end of the piston.

In one or more embodiments of the poultry processing systems including a piston pump assembly as described herein, when the piston driver is in the advanced position and the piston is in the rest position, the piston driver does not contact the trailing end of the piston such that a gap is present between the piston driver and the trailing end of the piston. In one or more embodiments, the assembly comprises a shim located between the piston driver and the trailing end of the piston, wherein the shim occupies the gap between the piston driver and the trailing end of the piston.

In one or more embodiments of the poultry processing systems including a piston pump assembly as described herein, the piston pump comprises a resting volume defined by a resting distance measured between the forward end of the piston and a terminal stop within the piston chamber, the terminal stop located between the chamber outlet and the forward end when the piston is in the rest position, wherein the selected height of the dosage puck is less than or equal to the resting distance.

In one or more embodiments of the poultry processing systems including a piston pump assembly as described herein, the piston pump comprises a resting volume defined by a resting distance measured between the forward end of the piston and a terminal stop within the piston chamber, the terminal stop located between the chamber outlet and the forward end when the piston is in the rest position, wherein the selected height of the dosage puck is less than the resting distance.

In one or more embodiments of the poultry processing systems including a piston pump assembly as described herein, the piston actuator comprises a fluid pressure actuator.

In one or more embodiments of the poultry processing systems including a piston pump assembly as described herein, the flow control apparatus comprises a check valve.

In one or more embodiments of the poultry processing systems including a piston pump assembly as described herein, the biasing apparatus comprises a spring.

In one or more embodiments of the poultry processing systems including a piston pump assembly as described herein, the chamber outlet is aligned with the piston axis.

In one or more embodiments of the poultry processing systems described herein, the system comprises: a first pump positioned on a first mount of a pumping base attached to the system platform, wherein the first mount comprises a first mount perimeter and a first plurality of actuation fluid delivery ports located within the first mount perimeter, wherein the first plurality of actuation fluid delivery ports defines a first delivery port pattern in the first mount perimeter; a second pump positioned on a second mount of the pumping base attached to the system platform, wherein the second mount comprises a second mount perimeter and a second plurality of actuation fluid delivery ports located within the second mount perimeter, wherein the second plurality of actuation fluid delivery ports defines a second delivery port pattern in the second mount perimeter, wherein the second delivery port pattern is the same as the first delivery port pattern; an actuation fluid delivery system configured to deliver actuation fluid to the first plurality of actuation fluid delivery ports and the second plurality of actuation fluid delivery ports; a first pump identification system configured to provide a first signal identifying the first pump; a second pump identification system configured to provide a second signal identifying the second pump; a controller operably connected to the actuation fluid delivery system, the first pump identification system, and the second pump identification system, wherein the controller is configured to: receive the first signal and the second signal; operate the actuation fluid delivery system to deliver actuation fluid to selected delivery ports of the first plurality of actuation fluid delivery ports at selected times based at least in part on the first signal; and operate the actuation fluid delivery system to deliver actuation fluid to selected delivery ports of the second plurality of actuation fluid delivery ports at selected times based at least in part on the second signal.

In one or more embodiments of the poultry processing systems including first and second pumps on a pumping base as described herein, the first pump identification system comprises a first identifier and a first reader, wherein the first reader is configured to read the first identifier and emit a selected first signal indicative of a pump type for the first pump. In one or more embodiments, the first identifier is attached to the first pump and the first reader is attached to the pumping base; or the first reader is attached to the first pump and the first identifier is attached to the pumping base.

In one or more embodiments of the poultry processing systems including first and second pumps on a pumping base as described herein, the second pump identification system comprises a second identifier and a second reader, wherein the second reader is configured to read the second identifier and emit a selected second signal indicative of a pump type for the second pump. In one or more embodiments, the second identifier is attached to the second pump and the second reader is attached to the pumping base; or the second reader is attached to the second pump and the second identifier is attached to the pumping base.

In one or more embodiments of the poultry processing systems including first and second pumps on a pumping base as described herein, the pump type is selected from the group of a piston pump and a peristaltic pump.

In one or more embodiments of the poultry processing systems including first and second pumps on a pumping base as described herein, when the first pump comprises a piston pump, the controller is configured to operate the actuation fluid delivery system to deliver actuation fluid to a first set of selected delivery ports of the first plurality of actuation fluid delivery ports; wherein, when the first pump comprises a peristaltic pump, the controller is configured to operate the actuation fluid delivery system to deliver actuation fluid to a second set of selected delivery ports of the first plurality of actuation fluid delivery ports; and wherein the first set and the second set comprise different sets of the first plurality of actuation fluid delivery ports.

In one or more embodiments of the poultry processing systems including first and second pumps on a pumping base as described herein, the first pump comprises a piston pump and the second pump comprises a peristaltic pump; the injection station comprises a dual port delivery chamber comprising a first inlet port, a second inlet port, and a needle port, wherein the first inlet port and the second inlet port are in fluid communication with the needle port and the injection needle is in fluid communication with the needle port; wherein the first pump comprises a piston pump in fluid communication with the first inlet port and the second pump comprises a peristaltic pump in fluid communication with the second inlet port; wherein the controller is configured to operate the actuation fluid delivery system to deliver actuation fluid to a first set of selected delivery ports of the first plurality of actuation fluid delivery ports; wherein the controller is configured to operate the actuation fluid delivery system to deliver actuation fluid to a second set of selected delivery ports of the second plurality of actuation fluid delivery ports; and wherein the first set of the first plurality of actuation fluid delivery ports defines a first selected pattern on the first mount and the second set of the second plurality of actuation fluid delivery ports defines a second selected pattern on the second mount, wherein the first selected pattern on the first mount and the second selected pattern on the second mount are different patterns.

In a second aspect, one or more embodiments of a method of processing poultry in a poultry processing system including a system platform and a support ring mounted on a base, the system platform comprising an injection sector comprising a plurality of mounting sites, a bird support mounted on the support ring outside of a perimeter of the system platform, an injection station attached to the system platform at a selected mounting site, the injection station comprising a needle assembly comprising an injection needle and a needle guide, the injection needle configured to move between an injection position and a retracted position along an injection axis, wherein a distal end of the injection needle extends out of a delivery site of the needle guide along the injection axis when the injection needle is in the injection position, and wherein the needle assembly and the needle guide are mounted on the injection carriage, the method comprises: rotating one or both of the system platform and the support ring relative to each other about a platform axis extending through the system platform, the support ring, and the base, wherein the rotating moves the bird support into and out of alignment with the selected mounting site; rotating the injection carriage about a carriage axis between a standby position and an actuation position, wherein the injection axis defines a base approach angle relative to the platform axis when the injection carriage is in the actuation position such that the injection needle approaches a bird support positioned at the selected mounting site at the base approach angle; and positioning a shim between the injection station and the selected mounting site such that, when the injection carriage is in the actuation position, the injection axis approaches the bird support at an adjusted approach angle relative to the platform axis.

In one or more embodiments of a method processing poultry as described herein, the method includes: positioning the shim between the injection station and the selected mounting site rotates the injection axis about the delivery site of the needle guide such that the delivery site of the needle guide is positioned at the same location relative to the platform axis when the injection axis is in the base approach angle and when the injection axis is in the adjusted approach angle. In one or more embodiments, the shim rotates the injection axis about the delivery site 2 degrees or more, 5 degrees or more, 10 degrees or more, 15 degrees or more, or 20 degrees or more, and, optionally, 30 degrees or less, 25 degrees lor less, 20 degrees or less, 15 degrees or less, or 10 degrees or less.

In one or more embodiments of a method processing poultry as described herein, the system comprises a piston pump assembly in fluid communication with the injection needle of the injection station, wherein the piston pump assembly comprises: a piston pump comprising a piston movable in a piston chamber of a housing defining a piston chamber, wherein movement of the forward end of the piston along a piston axis within the piston chamber forces fluid in the piston chamber out of the piston chamber through a chamber outlet, and a piston driver configured to be positioned proximate a trailing end of the piston when the piston driver is in the advanced position and the piston is in a rest position, and the method comprises: determining a selected volume of fluid to deliver to the injection needle from the piston pump; locating a selected dosage puck between the trailing end of the piston and the piston driver to move the piston driver away from the advance position, wherein the selected dosage puck comprises a selected height that corresponds to the selected volume in the pump chamber; and moving the piston driver to the advance position after locating the selected dosage puck between the trailing end of the piston and the piston driver to move the piston driver away from the advance position, wherein moving the piston driver to the advance position moves the piston towards the chamber outlet over a stroke distance equal to the selected height of the selected dosage puck to move the selected volume of fluid out of the piston chamber through the chamber outlet. In one or more embodiments, the method comprises, before locating the selected dosage puck between the trailing end of the piston and the piston driver: determining when a gap is present between the piston driver and the trailing end of the piston when the pump driver is in the advanced position and the piston is in the rest position; and locating a selected shim between the piston driver and the trailing end of the piston, wherein the selected shim occupies the gap between the piston driver and the trailing end of the piston. In one or more embodiments, the gap between the piston driver and the trailing end of the piston when the pump driver is in the advanced position and the piston is in the rest position comprises a gap height measured along the piston axis, and wherein the selected shim comprises a shim height that is equal to or greater than the gap height. In one or more embodiments, the piston pump comprises a resting volume defined by a resting distance measured between the forward end of the piston within the piston chamber when the piston is in the rest position, wherein the selected height of the dosage puck is less than or equal to the resting distance. In one or more embodiments, the piston pump comprises a resting volume defined by a resting distance measured between the forward end of the piston within the piston chamber when the piston is in the rest position, wherein the selected height of the dosage puck is less than the resting distance.

In one or more embodiments of a method processing poultry as described herein, the method comprises limiting rotational velocity of the injection carriage during rotation of the injection carriage from the standby position to the actuation position. In one or more embodiments, limiting the rotational velocity of the injection carriage comprises operably connecting a carriage damper to the injection carriage.

If used herein, relational terms such as above, below, top, bottom, etc. are (unless otherwise specified in this description and/or the claims) used only to facilitate description of the various features of the systems and methods described herein and should not be construed to require any specific orientation of the systems and/or the methods described herein.

If used herein, the term “substantially” has the same meaning as “significantly,” and can be understood to modify the term that follows by at least about 75%, at least about 90%, at least about 95%, or at least about 98%. The term “not substantially” as used herein has the same meaning as “not significantly,” and can be understood to have the inverse meaning of “substantially,” i.e., modifying the term that follows by not more than 25%, not more than 10%, not more than 5%, or not more than 2%.

Numeric values used herein include normal variations in measurements as expected by persons skilled in the art and should be understood to have the same meaning as “approximately” and to cover a typical margin of error, such as +5% of the stated value.

Terms such as “a,” “an,” and “the” are not intended to refer to only a singular entity but include the general class of which a specific example may be used for illustration.

The terms “a,” “an,” and “the” are used interchangeably with the term “at least one.” The phrases “at least one of′ and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure, including the claims.

The above summary of the invention is not intended to describe each embodiment or every implementation of the systems, apparatus, and methods described herein. Rather, a more complete understanding of the invention will become apparent and appreciated by reference to the following description of illustrative embodiments and claims in view of the accompanying figures of the drawing.

### BRIEF DESCRIPTION OF THE DRAWING

FIG. 1 is a top view of one illustrative embodiment of a poultry processing system as described herein.

FIG. 2 it is a schematic diagram of a side view of the illustrative embodiment of the poultry processing system depicted in FIG. 1.

FIG. 3 is a side elevation view of a bird support with a bird B located therein.

FIG. 4 is a perspective view of one illustrative embodiment of a poultry processing system including fluid delivery apparatus and a pair of injection stations mounted on a system platform.

FIG. 5 is a top view of a portion of the poultry processing system depicted in FIG. 4.

FIG. 6 is an enlarged perspective view of one of the injection stations depicted in FIGS. 4-5.

FIG. 7 is a perspective view of the support apparatus of the injection station depicted in FIG. 6 with the injection carriage removed from the injection station.

FIG. 8 is a perspective view of the injection carriage of the injection station depicted in FIG. 6 removed from the support apparatus.

FIG. 9 is an enlarged view of the needle mount and needle guide of the injection carriage depicted in FIG. 8.

FIG. 10 is a side view of a portion of the injection station depicted in FIG. 6 with an injection needle mounted on the needle mount of injection carriage and the injection carriage in an actuation position with respect to a bird support of a poultry processing system as described herein.

FIG. 11 is a side view of another illustrative embodiment of an injection station as described herein with an injection carriage in its standby position.

FIG. 12 depicts the injection station of FIG. 11 after movement of the injection carriage from the standby position to its actuation position as described herein.

FIG. 13 depicts the injection station of FIG. 12 (with the injection carriage in its actuation position) with the injection station rotated about a delivery site using a shim as described herein.

FIG. 14 is an enlarged front perspective view of one of the injection stations depicted in FIGS. 4-5.

FIGS. 15-16 are enlarged views of the illustrative embodiment of the skin capture assembly provided on the injection station of FIG. 14.

FIG. 17 is a perspective view of the support apparatus of the injection station depicted in FIG. 14.

FIG. 18 is a left side view of the injection station depicted in FIG. 14 with the support arm 137-1 of the support apparatus removed to expose components on the injection carriage of the injection station.

FIG. 19 is a perspective view of one of the injection stations depicted in FIGS. 4-5.

FIG. 20 is a side view of the injection carriage of the injection station depicted in FIG. 19 removed from the support apparatus of the injection station along with a dual port needle holder as described herein.

FIG. 21 is an enlarged top view of the dual port needle holder depicted in FIG. 20.

FIG. 22 is an enlarged front view of the dual port needle holder depicted in FIG. 20.

FIG. 23 is a perspective view of the poultry processing system depicted in FIG. 4 with one of the injection stations removed for clarity.

FIG. 24 is an enlarged top view of the poultry processing system as depicted in FIG. 23.

FIG. 25 is a perspective view of the pump base depicted in connection with the poultry processing system of FIGS. 23-24.

FIG. 26 is a top view of the pump base depicted in FIG. 25.

FIG. 27A is a view of the bottom of the piston pump assembly depicted in FIGS. 23-24.

FIG. 27B is a view of the bottom of the peristaltic pump assembly depicted in FIGS. 23-24.

FIG. 28 is a perspective view of a blanking plate that may be used on a pumping base as depicted in connection with the poultry processing system of FIGS. 23-24.

FIG. 29 is a schematic block diagram of a control system that may be used to control actuation of the pump assemblies depicted in connection with the poultry processing system of FIGS. 23-24.

FIG. 30 is a perspective view of one illustrative embodiment of a piston pump assembly that may be used in one or more embodiments of a poultry processing system as described herein.

FIG. 31 is a side view of one illustrative embodiment of a piston pump that may be used in a piston pump assembly of a poultry processing system as described herein.

FIG. 32 is a cross-sectional view of the piston pump depicted in FIG. 31 taken in a plane containing the piston axis 181.

FIG. 33 is a perspective view of the piston pump assembly of FIG. 30 with one of the piston pumps removed.

FIG. 34 is a view of the piston pump assembly of FIG. 33 with a dosage puck 206 located on a piston driver 202.

FIG. 35 is a cross-sectional view of one illustrative embodiment of a piston actuator position proximate a terminal end of one illustrative embodiment of a piston pump as described herein, with the piston driver in its advanced position and the piston of the piston pump in its rest position as described herein.

FIG. 36 is a cross-sectional view of the piston pump of FIG. 35 after positioning of a dosage puck between the piston and the piston driver and the piston driver in a retracted position as described herein.

FIG. 37 is a cross-sectional view of the piston pump of FIG. 36 after movement of the piston driver to its advanced position and corresponding movement of the piston as described herein.

FIG. 38 is a schematic block diagram of one illustrative embodiment of a control system that may be used to control piston actuators of a piston pump assembly of a poultry processing system as described herein.

FIGS. 39A-39B depicted one illustrative embodiment of a set of dosage pucks that may be used in one or more embodiments of a piston pump of a poultry processing system as described herein.

FIG. 40 is a perspective view of a portion of a piston pump assembly including shims used to adjust positions and dosage control of piston pumps as described herein.

FIGS. 41-45 are schematic diagrams depicting the use of dosage pucks and shims in one or more embodiments of piston pumps used in poultry processing systems as described herein.

While the above-identified figures (which may or may not be drawn to scale) set forth embodiments of the invention, other embodiments are also contemplated, as noted in the discussion. In all cases, this disclosure presents the invention by way of representation and not limitation. It should be understood that numerous other modifications and embodiments can be devised by those skilled in the art, which fall within the scope of this invention.

### DESCRIPTION OF ILLUSTRATIVE EMBODIMENTS

In the following description, reference is made to the accompanying figures of the drawing which form a part hereof, and in which are shown, by way of illustration, specific embodiments. It is to be understood that other embodiments may be utilized and changes may be made without departing from the scope of the present invention.

Illustrative embodiments of a poultry processing system, bird supports that may be used in the poultry processing system, and methods of using poultry processing system as well as various components that may be used in the poultry processing systems supports are described herein.

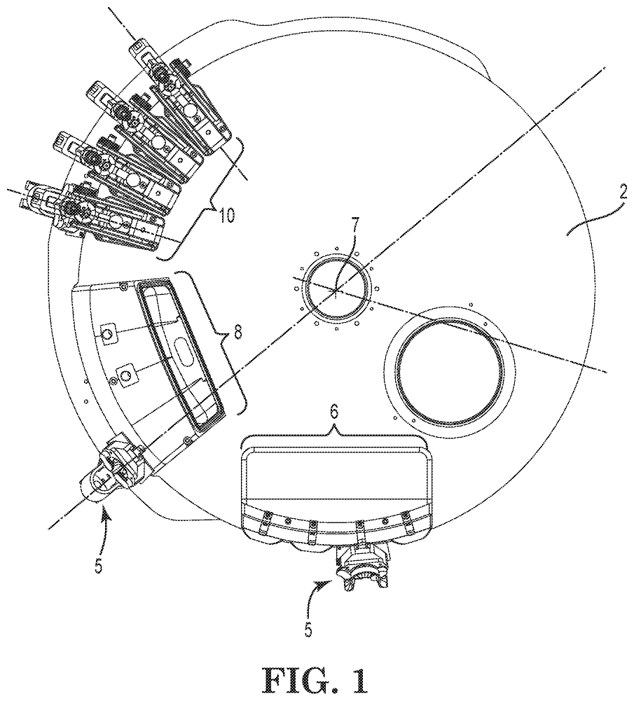

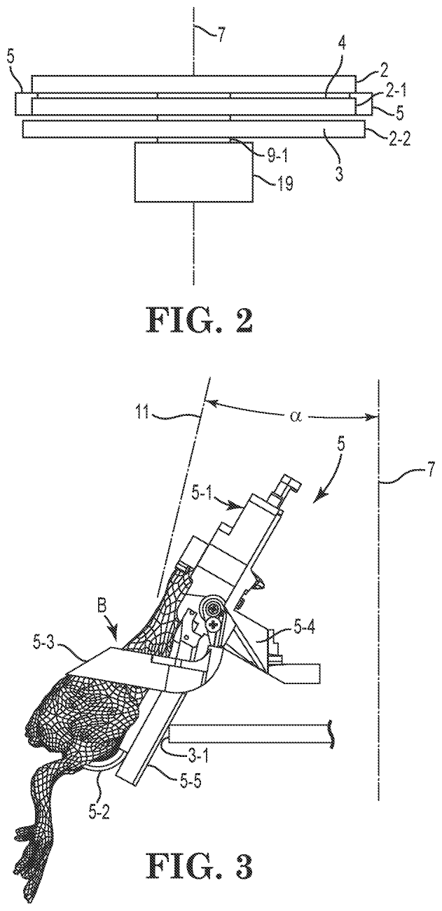

With reference to FIGS. 1-2, one illustrative embodiment of a poultry processing system as described herein may include a system platform 2, optional cam 3, support ring 4, and a plurality of bird supports 5 mounted on the support ring 4. In particular, it should be understood that the support ring 4 is visible in the schematic diagram of FIG. 2 but is obscured by the system platform 2 in the top plan view of FIG. 1.

The system platform 2 is, in one or more embodiments, typically fixed relative to the platform axis 7 such that the rotational position of the system platform 2 and any processing stations located thereon is also preferably fixed relative to the platform axis 7. In the depicted illustrative embodiment, the system platform 2 carries a set of loading stations 6 at which bird supports 5 are loaded with birds as described herein.

The system platform 2 depicted in FIG. 1 also carries a set of optional beak treatment stations 8, as well as a set of injection stations 10. In one or more embodiments, it may be preferable, but not required, to match the number of loading stations 6 with the number of beak treatment stations 8 and injection stations 10 (for example, in the depicted embodiment, the system platform 2 carries four loading stations 6, four beak treatment stations 8, and four injection stations 10). Although the depicted illustrative embodiment of poultry processing system includes beak treatment stations in addition to injection stations, other types of processing stations may be used in one or more embodiments of poultry processing systems as described herein in addition to and/or in place of the depicted beak treatment stations. Such alternative processing stations may include, for example, claw treatment stations, sexing or gender identification stations, etc.

Nonlimiting examples of processes and/or processing stations that may be used in one or more embodiments of the hatchling support systems described herein may be described in, for example, U.S. Pat. No. 5,195,925 (METHOD AND APPARATUS FOR DECLAWING POULTRY); U.S. Pat. No. 5,651,731 (METHOD AND APPARATUS FOR DEBEAKING POULTRY); U.S. Pat. No. 7,232,450 (APPARATUS AND METHOD FOR UPPER AND LOWER BEAK TREATMENT); U.S. Pat. No. 8,499,721 (APPARATUS AND METHOD FOR NASAL DELIVERY OF COMPOSITIONS TO BIRDS); U.S. Pat. No. 7,363,881 (BEAK TREATMENT WITH TONGUE PROTECTION); U.S. Pat. No. 9,775,695 (FOURTH TOE PROCESSING SYSTEMS AND METHODS); US Patent Application Publication US 2019/0133734 (POULTRY INJECTION APPARATUS WITH ROTATING CAPTURE MEMBERS AND METHODS OF USE); International Publication WO 2018204572 (INJECTION SYSTEMS AND METHODS); International Publication WO 2019236964 (ENERGY DELIVERY SYSTEM USING AN ELECTRIC FIELD); etc.

The number of processing stations of each type provided on any system platform used in a poultry processing system as described herein may vary depending on the timing required for processing birds at each of the processing stations. In one or more embodiments, the number of processing stations provided in different sets of processing stations of a poultry processing system as described herein may be the same. For example, in the depicted illustrative embodiment of a poultry processing system, the sets of loading stations 6, beak treatment stations 8, and injection stations 10 each include four stations. Sets of four stations in the depicted illustrative embodiment of poultry processing system may be beneficial for systems in which birds are manually loaded into bird supports in the loading stations 6. In alternative embodiments of poultry processing systems as described herein, two or more different sets of processing stations may, however, include different numbers of processing stations.

In one or more embodiments, the support ring 4 is mounted on a base 9 using a central support assembly 9-1, with the support ring 4 being configured for rotation about a platform axis 7 that, in the depicted illustrative embodiment, extends through each of the system platform 2 and cam 3. The base 9 may include any suitable drive mechanism (for example, electric motor, hydraulic motor, etc.) that may be used to rotate the support ring 4 about the platform axis 7 as needed to move the bird supports 5 through the poultry processing system as described herein.

As described herein, the bird supports 5 are, in one or more embodiments, mounted on a periphery of the support ring 4. Each bird support 5 is mounted in a fixed location on the periphery of the support ring 4. As a result, rotation of the support ring 4 about the platform axis 7 rotates the bird supports 5 about the platform axis 7.

Although only two bird supports 5 are depicted in the top plan view of FIG. 1 and the schematic side elevation view of FIG. 2, poultry processing systems as described herein will typically include bird supports located about a majority, if not substantially all, of the periphery of the support rings of the poultry processing systems. The bird supports 5 will typically be provided in spacing about the periphery of the support ring 4 that matches the spacing needed to properly locate birds in bird supports 5 at any processing stations (for example, beak treatment stations 8 and/or injection stations 10) provided in the poultry processing systems described herein.

The illustrative embodiments of poultry processing systems described herein include bird supports and an optional cam used to move the bird supports between open and closed configurations as described in, e.g., International Publication WO 2021/150842 titled HATCHLING SUPPORTS, HATCHLING SUPPORT SYSTEMS, AND METHODS. Although the illustrative embodiments of bird supports as specifically described herein may be useful in combination with the poultry processing systems described herein, alternative bird supports may also be used with the poultry processing systems and methods described herein. Potentially useful alternative bird supports that may be used in the systems and methods described herein may include, e.g., those described in U.S. Pat. No. 9,808,328 titled POULTRY CARRIERS AND METHODS OF RESTRAINING POULTRY and other publications.

With reference to FIG. 3, one bird support 5 similar to those described in International Publication WO 2021/150842 is depicted with a bird B located in the bird support. The bird support 5 includes a head holder 5-1, cradle 5-2 and calming band 5-3 attached to a base 5-4 used to attach the bird support 5 to a support ring 4 of a poultry processing system as described herein. As described in International Publication WO 2021/150842, the depicted embodiment of bird support 5 includes a follower 5-5 that is configured to contact the peripheral edge 3-1 of the cam 3. Contact between the followers of the bird supports 5 and the peripheral edge 3-1 of the cam 3 is configured to hold the followers of the bird supports 5 in a closed position in at least one section of the cam 3 with the bird support 5 of FIG. 3 being in the closed configuration.

Regardless of the specific form of the bird supports used in the poultry processing systems described herein, i.e., whether the bird supports are similar to those described in International Publication WO 2021/150842, U.S. Pat. No. 9,808,328, or other alternatives, the bird supports have a fixed orientation relative to, e.g., the platform axis 7 or other stationary features of the poultry processing systems described here and, as a result, the bird supports present birds retained in the bird supports to the processing stations of the poultry processing systems at a fixed orientation and position to facilitate processing. As depicted in FIG. 3, for example, the bird support 5 is attached to support ring 4 at an orientation that is fixed relative to the platform axis 7 and also relative to an injection axis 11 of an injection station 10.

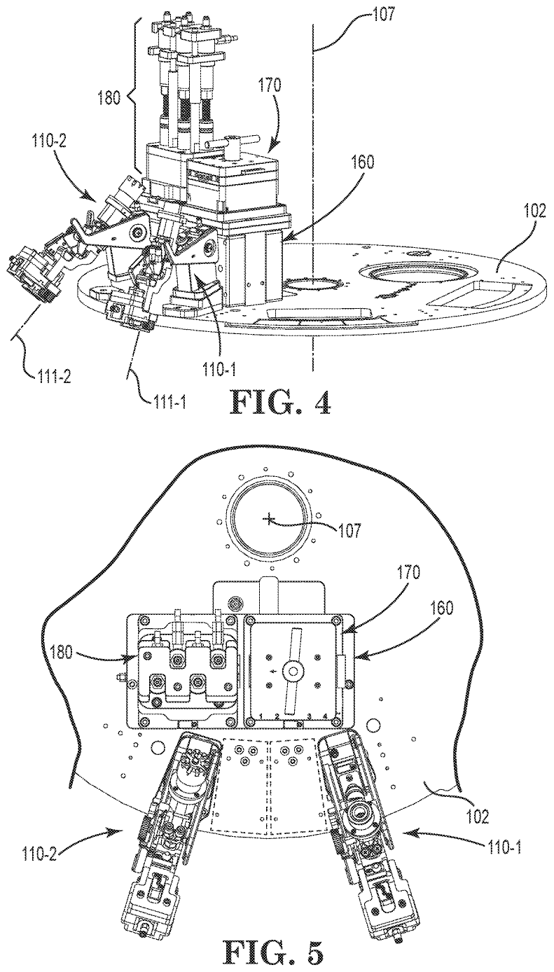

FIG. 4 is a perspective view and FIG. 5 is a top view of one illustrative embodiment of a poultry processing system as described herein including a pair of injection stations 110-1 and 110-2 (collectively referred to herein as injection stations 110) mounted on a system platform 102. Although the depicted poultry processing system includes two injection stations 110, the system platform 102 includes space for a set of four injection stations similar to those depicted in the illustrative embodiment, with the two depicted injection stations 110 being located on opposite ends of the injection section and space for two additional injection stations mounted on the system platform 102 between the two depicted injection stations 110.

Each of the injection stations 110 depicted in FIGS. 4 and 5 defines an injection axis 111-1 or 111-2 (collectively referred to herein as injection axes 111) that is an axis along which an injection needle in each of the injection stations 110 advances during processing of a bird. Also depicted is the platform axis 107 about which bird supports mounted on a support ring (neither of which is shown in FIGS. 4 and 5) rotate to bring the bird supports into alignment with the injection stations as described herein.

Also mounted on the system platform 102 is a pump base 160 on which a pair of pump assemblies are mounted. The pump assemblies include a peristaltic pump assembly 170 and a piston pump assembly 180. The pump assemblies 170/180 are used to deliver fluids to the injection stations of the poultry processing system as described herein.

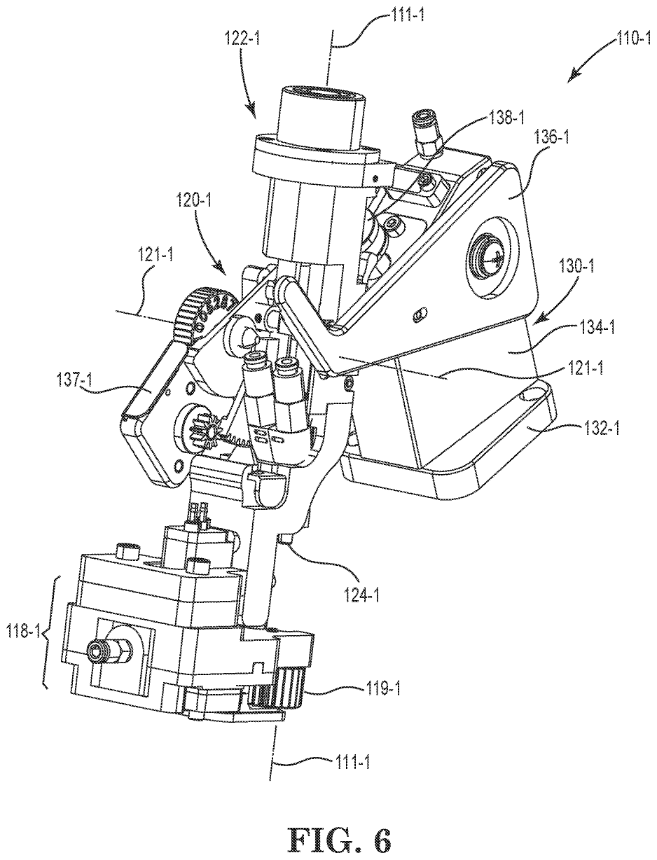

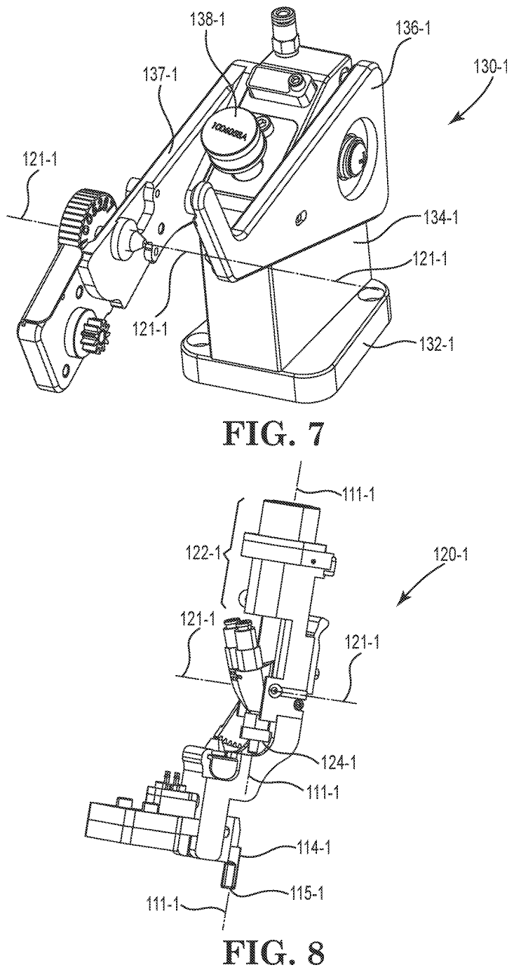

FIGS. 6-8 depict one of the illustrative embodiments of injection stations 110 depicted in FIGS. 4 and 5. In particular, FIG. 6 is a perspective view of the injection station 110-1 while FIG. 7 is a perspective view of the support apparatus 130-1 and FIG. 8 is a perspective view of the injection carriage 120-1 removed from support apparatus 130-1. The injection carriage 120-1 is mounted on the support apparatus 130-1 for rotation about a carriage axis 121-1. In particular, the support apparatus includes a base 132-1, tower 134-1, and support arms 136-1 and 137-1, with the injection carriage 120-1 supported directly by the support arms 136-1 and 137-1 for rotation about carriage axis 121-1. Also depicted in FIGS. 6 and 7 is a carriage actuator 138-1 which acts on the injection carriage 120-1 to rotate the injection carriage 120-1 about the carriage axis 121-1.

The injection carriage 120-1 includes a depth control adjustment mechanism 122-1 (see, e.g., FIGS. 6 and 8) used to control the travel and, therefore, the depth of any injection performed using the poultry processing systems as described herein. Although not required, depth control mechanism 122-1 may be similar or operate under similar concepts as those described in connection with the injection systems discussed in US Patent Application Publication US 2020/0155289. Further, the actuators used to advance and retract an injection needle along the injection axis 111-1 in the injection stations described herein may, in one or more embodiments, also be similar to those discussed in the above reference.

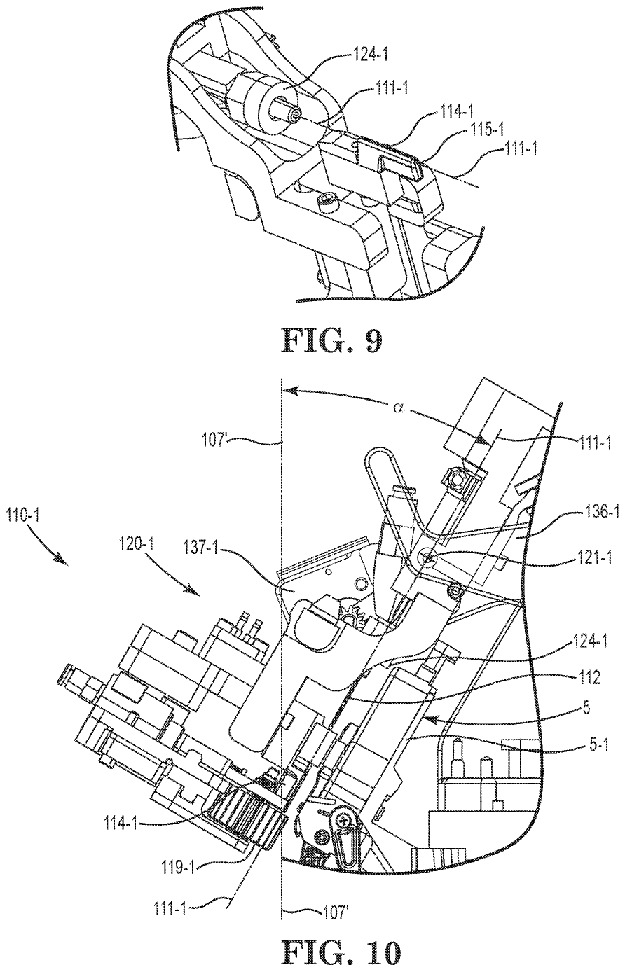

The injection carriage depicted in FIGS. 6-9 does not include an installed injection needle. The injection carriage 120-1 does, however, include an injection needle mount 124-1 as well as a needle guide 114-1 used to guide and control movement of an injection needle along the injection axis 111-1. FIG. 9 is a bottom back side perspective view of the injection needle mount 124-1 and needle guide 114-1 of injection carriage 120-1 depicting injection axis 111-1 as extending from the needle mount 124-1 through the needle guide 114-1 to a delivery site 115-1 on needle guide 114-1 through which an injection needle extends when mounted on the injection carriage 120-1. In one or more embodiments, the depicted injection needle mount 124-1 may be in the form of a Luer fitting that mates with a complementary Luer fitting on an injection needle to retain an injection needle in position in the depicted injection station. Other embodiments of needle mounts may use alternative connection apparatus suitable to attach an injection needle in the injection carriages of injection stations of poultry processing systems described herein.

FIG. 10 depicts a portion of the injection station 110-1 as depicted in FIGS. 6-9 with an injection needle 112 mounted on the needle mount 124-1 of injection carriage 120-1 with the injection needle 112 passing through the needle guide 114-1 along injection axis 111-1. The injection station 110-1 of FIG. 10 is attached to the system platform 102 with a head restraint 5-1 of a bird support 5 aligned with the injection station 110-1. The bird support 5 and its associated head restraint 5-1 are similar to those depicted in FIG. 3. As discussed herein, the injection station 110-1 includes a needle assembly including an injection needle 112 and a needle guide 114-1. The injection needle 112 is configured to move between an injection position and a retracted position along the injection axis 111-1. In particular, a distal end of the injection needle 112-1 extends out of a delivery site 115-1 of the needle guide 114-1 along the injection axis 111-1 when the injection needle is in the injection position so that the injection needle 112 can be delivered subcutaneously into a bird retained in the bird support 5.

In the depicted embodiment of injection station 110-1, an optional skin capture assembly 118-1 is provided proximate the delivery site of the needle guide 114-1, with the skin capture assembly 118-1 including one or more rotating capture members 119-1 configured to capture or bunch the skin of a bird located in the bird support 5 to facilitate the injection process. Similar apparatus are described in, e.g., U.S. Pat. No. 10,813,735.

Also depicted in FIG. 10 is a translated platform axis 107′, with the translation occurring in the plane defined by rotation of the bird supports about the platform axis 107 such that the translated platform axis 107′ retains the same orientation relative to the system platform 102 and other stationary components of the poultry processing systems described herein. The translated platform axis 107′, as depicted in FIG. 10, is positioned so that it intersects the injection axis 111-1 at the delivery site 115-1 in needle guide 114-1.

As depicted in FIG. 10 the injection carriage of the injection station 110-1 is in an actuation position as described herein. When in the actuation position, the injection axis 111-1 defines an approach angle α (alpha) relative to the translated platform axis 107′ and is positioned to be delivered subcutaneously into a bird retained in the bird support 5.

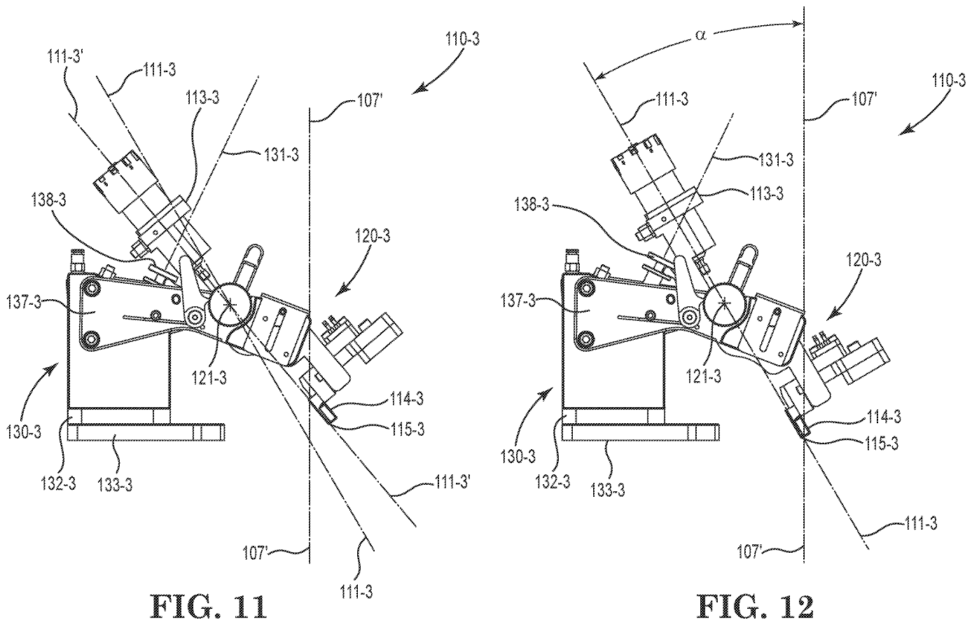

FIGS. 11-12 depict another illustrative embodiment of an injection station 110-3 that is similar to the injection stations 110 as seen in FIGS. 4-5 and which is, with the exception of the features used to mount an injection needle in the injection station, constructed similarly to injection station 110-1. FIGS. 11-12 are provided to depict movement of the injection carriage 120-3 of injection station 110-3 between a standby position and an actuation position—with the injection carriages 120-1 and 120-2 of injection stations 110-1 and 110-2 (respectively) including the same components and operating in the same manner as the injection carriage 120-3 of injection station 110-3.

With reference to FIG. 11, the injection station 110-3 includes a support apparatus 130-3 which supports the injection carriage 120-3 for rotation about carriage axis 121-3 using a carriage actuator 138-3 as discussed herein. With reference to FIGS. 11-12, the depicted embodiment of carriage actuator 138-3 advances and retracts along actuator axis 131-3 to move the injection carriage 120-3 between its standby position as depicted in FIG. 11 and its actuation position as depicted in FIG. 12.

The depicted illustrative embodiment of carriage actuator 138-3 is only one example of a carriage actuator that may be used to move an injection carriage of an injection station between its standby position and actuation position in a poultry processing system as described herein. Examples of some potentially suitable actuators that may be used for the carriage actuators of one or more embodiments of injection stations used in one or more embodiments of poultry processing systems as described herein may include, e.g., hydraulic pistons, solenoids, etc. Any structure capable of providing the motion required to move the injection carriage as described herein could be used as an actuator. Some potentially suitable examples may include, e.g., reversible motors, reciprocating mechanisms, etc.

Although an injection needle is not depicted In FIGS. 11-12 in connection with the injection station 110-3, a needle actuator 113-3 is depicted in FIGS. 11-12 and is used to advance and retract an injection needle along the injection axis (e.g., injection axis 111-3) as described herein. Examples of some potentially suitable actuators that may be used for the needle actuators of one or more embodiments of injection stations as described herein may include, e.g., hydraulic pistons, solenoids, etc. Any structure capable of providing the motion required to move the injection needles as described herein could be used as an actuator. Some potentially suitable examples may include, e.g., reversible motors, reciprocating mechanisms, etc.

FIG. 11 depicts the injection carriage 120-3 in the standby position while the injection carriage 120-3 is in the actuation position in FIG. 12. Movement of the injection carriage between the standby position depicted in FIG. 11 and the actuation position depicted in FIG. 12 may be useful to provide additional clearance for movement of bird supports and birds retained therein into and out of alignment with the injection stations 110 as birds are moving through the poultry processing systems described herein.

When the injection carriage 120-3 is in the standby position as depicted in FIG. 11, the injection axis 111-3 is offset from its position when the injection carriage 120-3 is in the actuation position (see, for example, injection axis 111-1 of injection station 110-1 as depicted in FIG. 10). To illustrate the different positions of injection axis 111-3, both an injection axis 111-3′ illustrative of the position of the injection axis when the injection carriage 120-3 is in the standby position, and injection axis 111-3 illustrative of the position of the injection axis when the injection carriage 120-3 is in the actuation position (as seen in FIG. 12) are depicted in FIG. 11.

When the injection carriage 120-3 of the injection station 110-3 is in an actuation position as depicted in FIG. 12, the injection axis 111-3 defines an approach angle α (alpha) relative to the translated platform axis 107′ and is, as discussed in connection with FIG. 10, positioned to be delivered subcutaneously into a bird retained in the bird support 5.

Although an injection axis 111-3 defining an approach angle α (alpha) relative to the platform axis 107 may be useful in connection with some birds it may be beneficial to adjust or vary the approach angle, i.e., the angle formed between the injection axis and a platform axis in poultry processing systems in which bird supports (and birds contained therein) rotate about the platform axis to move into and out of alignment with injection stations as described herein. Although described relative to the platform axis, the approach angle also corresponds to the angle at which an injection needle approaches a bird support aligned with the injection station in one or more embodiments of the poultry processing systems as described herein.

Adjustment of the approach angle may provide a variety of benefits. For example, adjustment of the approach angle may provide for more consistent delivery of liquids through an injection needle based on bird anatomy. For example, variations in bird anatomy may result in inefficient or ineffective subcutaneous delivery of fluids through an injection needle if the approach angle is fixed and not adjustable. Anatomical differences found between different species of birds (e.g., chickens, ducks, turkeys, etc.) or even within the same species may make adjustment of the approach angle advantageous when setting up a poultry processing system as described herein.

Although a variety of techniques could be used to change or adjust approach angles on rotating injection carriages, e.g., adjusting stops used to control rotation of an injection carriage, adjusting the travel of a carriage actuator, etc., such changes may result in unwanted movement of the position of the delivery site of the injection needle.

One or more embodiments of the poultry processing systems described herein incorporate features that allow for adjustment of the approach angle of an injection needle as described herein without changing the position of the delivery site of the injection needle. During development of the injection stations and poultry processing systems described herein, it has been found that consistent positioning of the delivery site of the injection needle between different approach angles is beneficial because it avoids the need to change any positioning of the bird supports relative to the remainder of the poultry processing system such as, e.g., the platform axis, etc.

In one or more embodiments of the poultry processing systems described herein the approach angle of an injection needle of an injection station without changing the position of the delivery site of the injection needle can be accomplished using one or more shims positioned between a base of an injection station and the system platform.

Using shims positioned between the injection stations and the system platform to adjust the approach angles as described herein provides an additional advantage because the injection stations themselves may be uniform, i.e., require no additional adjustments, which can simplify manufacturing of the injection stations, set up of poultry processing systems using the injection stations, replacement of components of the injection stations, etc. Because shims have a fixed or set size and shape, the use of shims to adjust approach angles eliminates the need to make adjustments that might vary between injection stations due to, e.g., human error, slippage (due, e.g., to improperly tightened connectors), etc.

The use of shims to adjust approach angles is particularly useful in poultry processing systems in which bird supports have a fixed orientation relative to, e.g., the platform axes or other stationary features of the poultry processing systems described herein. Because the bird supports present birds retained in the bird supports to the processing stations of the poultry processing systems at a fixed orientation and position relative to stationary features such as, e.g., the injection stations, the approach angles defined by the injection axes of the injection stations are uniform when the injection stations are mounted on the system platforms using either no shims or the same shims.

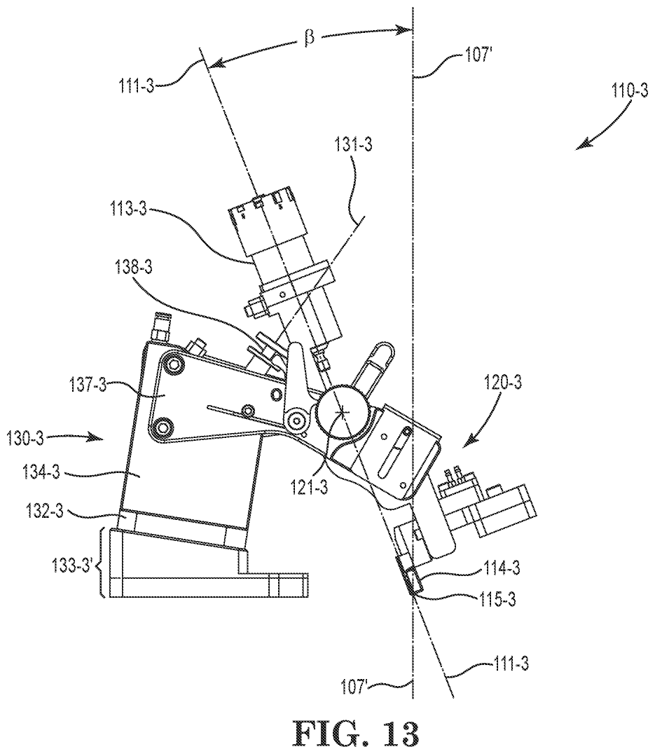

With reference to FIGS. 12-13, the use of shims to adjust approach angle without changing the position of the delivery site of the injection needle is illustrated. In particular, FIG. 12 depicts an injection station 110-3 in which the base 132-3 of the support apparatus 130-3 is positioned on a neutral shim 133-3 having a constant thickness as measured along the platform axis and, therefore, the translated platform axis 107′. As a result, the injection axis 111-3 defines an approach angle α (alpha) with the translated platform axis 107′. Further, the delivery site 115-3 defined in the needle guide 114-3 is located at the intersection between the translated platform axis 107′ and the injection axis 111-3. In use, neutral shim 133-3 would typically, but not necessarily, be located directly on the system platform 102 (see, e.g., a corner of a neutral shim positioned between the injection station 110-2 and system platform 102 in FIG. 4).

When used, neutral shims, such as neutral shim 133-3, simply raise the delivery site of an injection station along the direction of the platform axis. In one or more embodiments, shims may also be used to move the delivery site of an injection station towards or away from the platform axis as needed.

With reference to FIG. 13, a wedge-shaped shim 133-3′ rotates the injection axis 111-3 of the injection station 110-3 to form an adjusted approach angle β (beta) with the translated platform axis 107′ (and, therefore, platform axis 107) such that the injection needle approaches a bird support aligned with the injection station 110-3 at the adjusted approach angle.

Although the injection station 110-3 and its injection axis 111-3 are rotated by the shim 133-3′, the position of the delivery site of the injection needle as represented by the delivery site opening 115-3 in needle guide 114-3 remains constant relative to any selected fixed position within the poultry processing system.

Although wedge-shaped shim 133-3′ only rotates the injection axis 111-3 relative to neutral shim 133-3, one or more alternative embodiments of shims that may be used to rotate injection axes of injection stations in poultry processing systems as described herein may be used to both rotate the injection axes and raise or lower the position of the delivery sites relative to the platform axes if needed or desired.

Also, although described as being constant, the exact position of the delivery sites of the injection needles of injection stations of poultry processing systems may move slightly between different approach angles when shims are used to rotate the injection axes, but the positions of the delivery sites remain functionally constant relative to other fixed features of the poultry processing system such as, e.g., the platform axis, bird support positions, etc. By functionally constant, it is meant that the efficacy or effectiveness of the injection systems is not materially changed due to any such slight movements of the delivery sites between different approach angles.

Representative approach angles that may be useful in one or more poultry processing systems as described herein may range from, at the low end, 10 degrees or more, 12 degrees or more, 15 degrees or more, or 20 degrees or more. At the upper end, representative approach angles that may be useful in one or more poultry processing systems as described herein may range from 30° or less, 27° or less, 24° or less, 21° or less, 18° or less, or 15° or less. Variations in the approach angle may, however, be driven by the orientation of the bird supports and the birds located therein between different poultry processing systems and these representative angles are only one set of approach angles that may be useful in poultry processing systems as described herein.

Although described as rotating the approach angle, it must be understood that the position of the delivery site of the injection needle as the approach angle is rotated is depicted in the absence of a bird aligned with the injection station. The injections stations are typically designed such that a bird will occupy the location of the delivery site and can, itself, change the approach angle because the injection carriage will be prevented from reaching the position depicted in, e.g., FIGS. 12 and 13 due to the bird. Regardless, the shim 133-3′ of FIG. 13 would result in a different approach angle than that provided by, e.g., neutral shim 133-3 of FIG. 12.

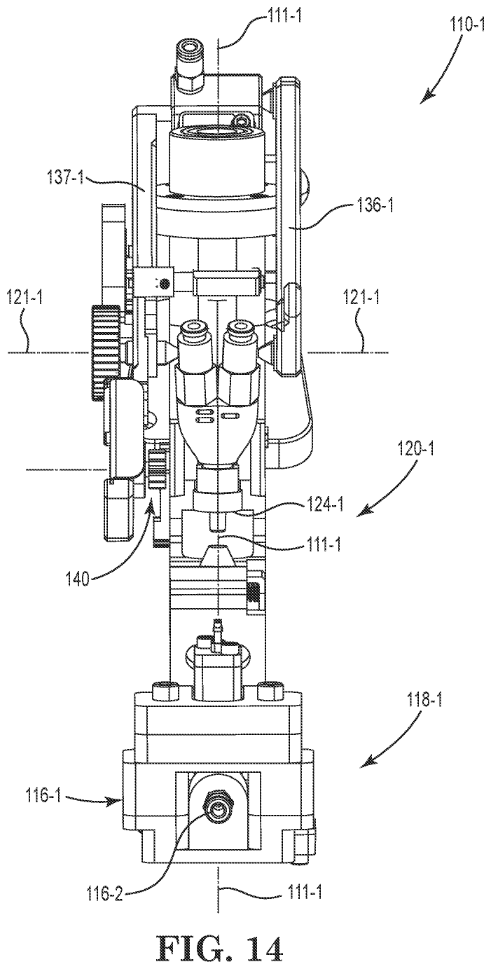

One or more embodiments of the injection stations of one or more embodiments of the poultry processing systems described herein may include a carriage damper to limit the velocity and, therefore, the impact energy that the injection carriage delivers to a bird as the injection carriage moves from the standby position to the actuation position. FIGS. 14-18 depict one illustrative embodiment of a carriage damper that may be used in the injection stations.

FIG. 14 is a top front perspective view of the injection station 110-1 depicted in FIGS. 4-6. The injection station 110-1 includes an injection carriage 120-1 that, as described herein, is configured to carry an injection needle for movement along an injection axis 111-1. The injection carriage 120-1 is supported by support arms 136-1 and 137-1 and rotates about carriage axis 121-1 between the support arms between a standby position and an actuation position as described in connection with, for example, injection station 110-3 as depicted in FIGS. 11-12.

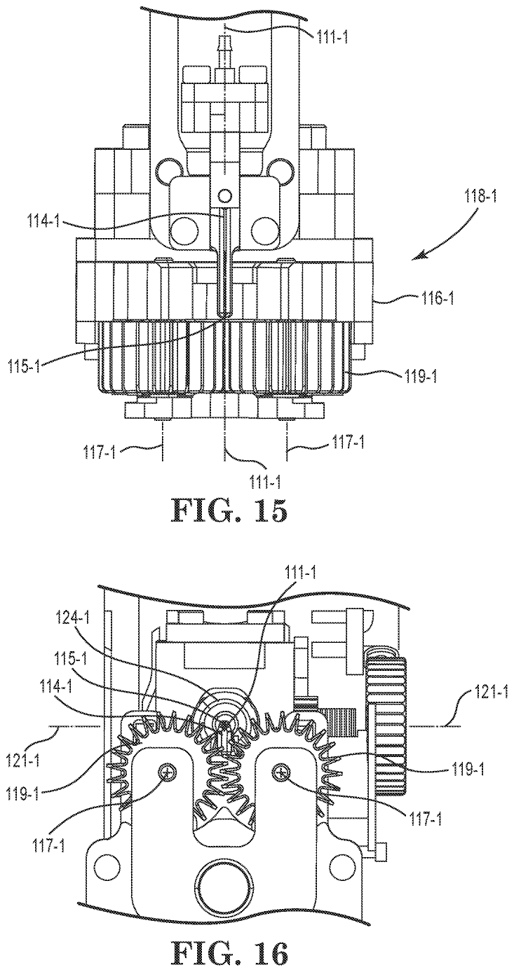

As discussed herein, the depicted embodiment of injection station 110-1 includes an optional skin capture assembly 118-1. FIG. 15 is an enlarged back side view of the skin capture assembly 118-1 while FIG. 16 is an enlarged view of the skin capture assembly 118-1 taken along the injection axis 111-1 from below the skin capture assembly 118-1 as depicted in FIG. 14. The skin capture assembly 118-1 includes capture members 119-1 configured to capture or bunch the skin of a bird located in the bird support 5 to facilitate the injection process as described in, e.g., U.S. Pat. No. 10,813,735. In the depicted illustrative embodiment, both of the capture members 119-1 are configured to rotate about their respective axes 117-1.

The delivery site 115-1 in needle guide 114-1 is positioned such that, as an injection needle attached to the needle mount 124-1 advances out of the delivery site 115-1 in needle guide 114-1 after operation of the skin capture assembly 118-1, the injection needle pierces skin bunched up by the rotating capture members 119-1.

The capture members 119-1 of skin capture assembly 118-1 are rotated by a drive system 116-1 contained at the end of the injection carriage 120-1. In the depicted illustrative embodiment, the drive system 116-1 is a pneumatically driven drive system with compressed air being provided at port 116-2 on the injection carriage 120-1.

Although the skin capture assembly 118-1 provides advantages in effective and repeatable subcutaneous delivery of an injection needle along injection axis 111-1, the mass of the skin capture assembly 118-1 is sufficient such that damping or control over the rotational velocity of the injection carriage 120-1 can limit the impact forces on a bird located in a bird support aligned with the injection station 110-1 as the injection carriage 120-1 moves into its actuation position as described herein.

In the depicted illustrative embodiment of injection station 110-1, control over the rotational velocity of the injection carriage 120-1 is provided by a carriage damper 140 as depicted in FIG. 14. In one or more embodiments, the carriage damper may be described as a rotary damper because it limits rotational velocity of the injection carriage 120-1 about carriage axis 121-1.

In one or more embodiments, it may be preferred that the carriage damper 140 is a one-way damper which controls rotational velocity of the injection carriage 120-1 when moving from the standby position to the actuation position but does not limit rotational velocity of the injection carriage 120-1 when moving in the opposite direction, i.e., from the actuation position to the standby position. Using a one-way damper may be beneficial because moving the injection carriage 120-1 away from a bird in a bird restrained faster (i.e., without damping) may allow for decreased (i.e., faster) cycle times as bird supports are moved into and out of alignment with the injection stations as described herein.

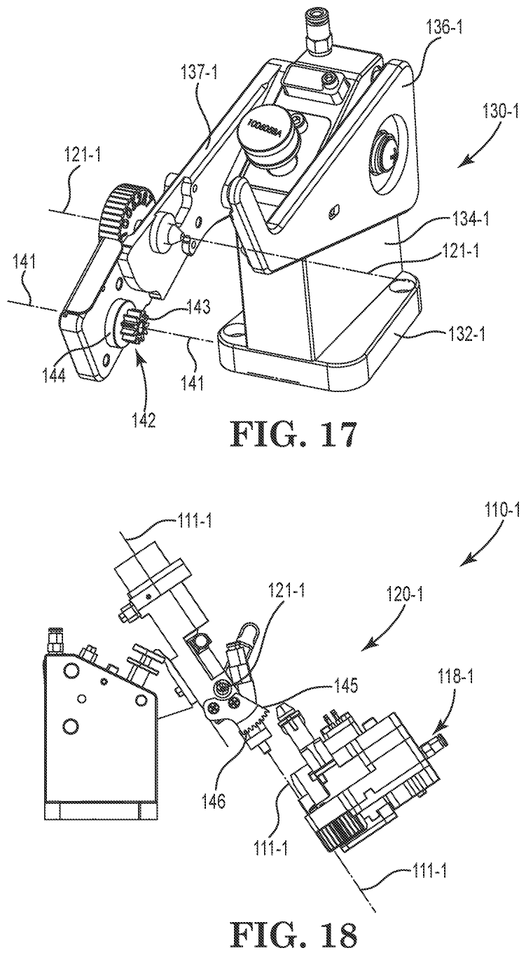

Components of the illustrative embodiment of carriage damper 140 are depicted separately in FIGS. 17-18. In particular, FIG. 17 depicts the support apparatus 130-1 of injection station 110-1 with the injection carriage 120-1 removed. With the injection carriage 120-1 removed from FIG. 17, the damper unit 142 is exposed. The depicted embodiment of damper unit 142 includes a gear 143 operably attached to a housing 144 that houses the damping apparatus. In particular, rotation of gear 143 about damping axis 141 is impeded. Such rotary damping apparatus are well-known and will not be further described herein.

With reference to FIG. 18, a complementary gear segment 145 is attached to the injection carriage 120-1 with the gear segment 145 including teeth 146 configured to mesh with the gear 143 on damper unit 142 when assembled in the injection station 110-1.

As the injection carriage 120-1 rotates about carriage axis 121-1 to move the injection carriage from its standby position to its actuation position, the components of the carriage damper 140 cooperate to limit or reduce the velocity of the injection carriage 120-1 and, thereby, reduce the impact energy exerted by the skin capture assembly 118-1 on a bird retained in a bird support aligned with the injection station 110-1.

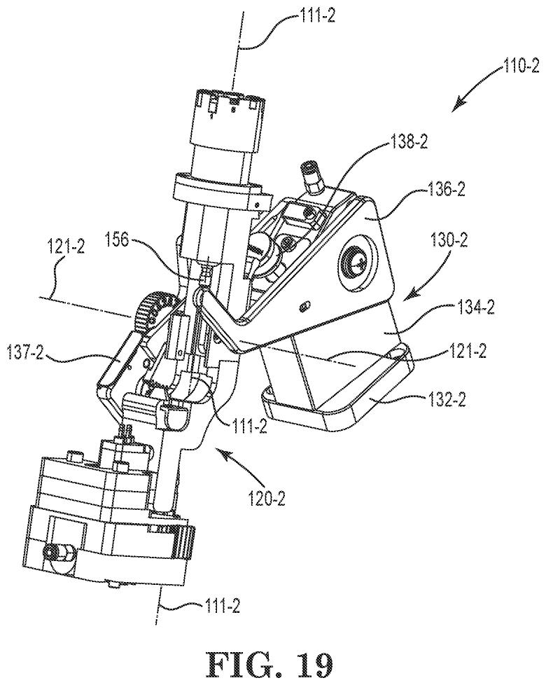

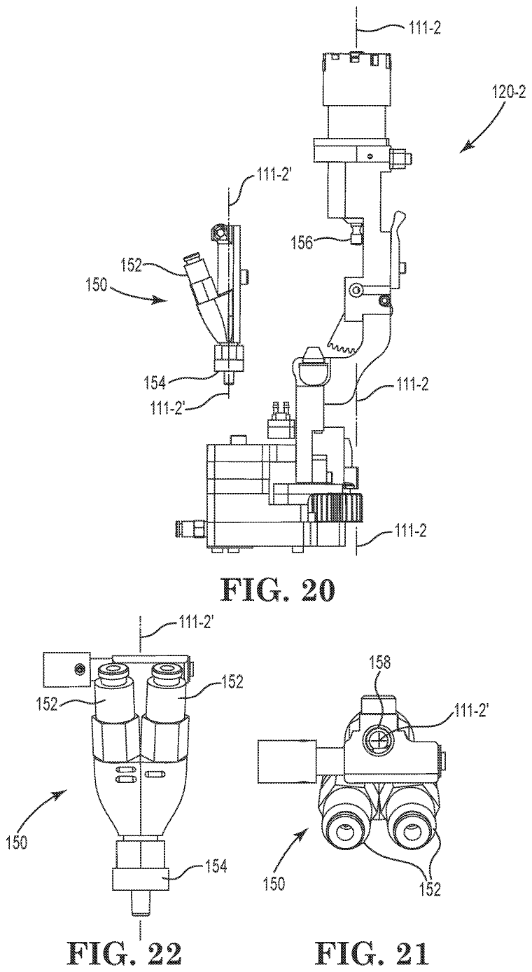

The illustrative embodiment of injection station 110-2 depicted in FIGS. 4-5 is depicted in an enlarged view in FIG. 19. The injection station 110-2 includes an injection carriage 120-2 operably attached to a support apparatus 130-2. The support apparatus includes a base 132-2, tower 134-2, and support arms 136-2 and 137-2, with the injection carriage 120-2 supported directly by the support arms 136-2 and 137-2 for rotation about carriage axis 121-2. Also depicted in FIG. 19 is a carriage actuator 138-2 which acts on the injection carriage 120-2 to rotate the injection carriage 120-2 about the carriage axis 121-2. A side view of the injection carriage 120-2 removed from the injection station 110-2 is depicted in FIG. 20 with the injection axis 111-2 depicted in both FIGS. 19-20.

The injection carriage 120-2 does not include an injection needle mount as seen in connection with the injection carriage 120-1 of injection station 110-1 described above. Injection carriage 120-2 does not include an injection needle mount because it is designed to receive a needle carrier that is, itself, configured to receive an injection needle.

One illustrative embodiment of a needle carrier 150 is seen in FIGS. 20-22. In particular, FIG. 20 includes a side view of the needle carrier 150 with an injection axis 111-2′ extending through needle carrier 150 to assist with understanding the location of the needle carrier 150 when properly inserted into the injection carriage 120-2 (it being understood that injection axis 111-2′ is coextensive with injection axis 111-2 extending through the injection carriage 120-2).

The needle carrier 150 includes a pair of ports 152 configured for connection to a fluid delivery system as well as an injection needle mount 154. In one or more embodiments, the depicted injection needle mount 154 may be in the form of a Luer fitting that mates with a complementary Luer fitting on an injection needle to retain an injection needle in position in the depicted injection station. Other embodiments of needle mounts may use alternative connection apparatus suitable to attach an injection needle to needle carriers used in the injection carriages of injection stations of poultry processing systems described herein.

The ports 152 of needle carrier 150 are in fluid communication with the injection needle mount 154 such that fluid delivered into the needle carrier 150 through either one of the ports 152 passes out of the needle carrier 150 through injection needle mount 154 for delivery to an injection needle attached to the needle carrier 150. In one or more embodiments, the needle carrier 150 may include one or more check valves or other fluid control components to control fluid flow through the needle carrier 150 as described in, e.g., International Publication No. WO 2021/211403 titled SINGLE NEEDLE SEQUENTIAL INJECTION SYSTEMS AND METHODS.

The depicted illustrative embodiment of needle carrier 150 includes a bore 158 (see, e.g., FIG. 21) while the injection carriage 120-2 includes a pin 156 sized and positioned to be received in the bore 158 of needle carrier 150 when needle carrier 150 is properly inserted into the injection carriage 120-2. When the needle carrier 150 is properly inserted into the injection carriage 120-2, the needle mount 154 is aligned along the injection axis 111-2 such that an injection needle attached to the needle mount 154 moves along injection axis 111-2 as described in connection with the poultry processing systems described herein.

The dual port needle carrier 150 depicted in FIGS. 20-22 is useful in poultry processing systems in which different fluids are to be delivered using a single injection needle. Typically, the different fluids would be delivered to the same bird in a bird support aligned with the injection station 110-2 although that is not required.

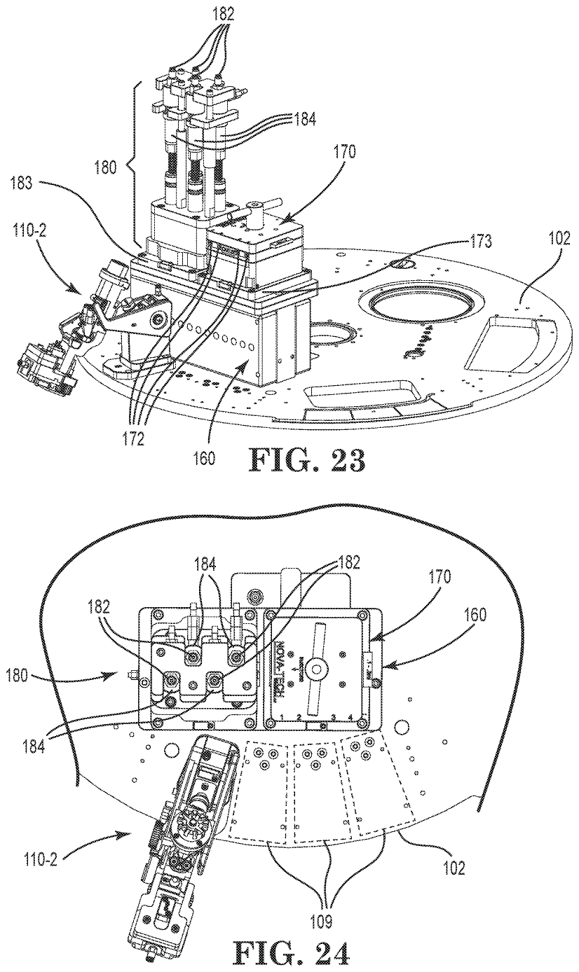

FIGS. 23-24 depict the injection station 110-2 in position on a system platform 102 as also depicted in, for example, FIGS. 4-5. Also depicted in FIGS. 23-24 are a pair of pumps used to deliver fluids to one or more of the injection stations located on system platform 102. The poultry processing system depicted in FIGS. 23-24 includes two different pump assemblies including a peristaltic pump assembly 170 and a piston pump assembly 180.

In one or more embodiments of the poultry processing systems described herein in which injection stations are provided to deliver different liquids through subcutaneous (or other) needle-based injection, delivery of different liquids having significantly different properties may require that those liquids be delivered at different pressures, temperatures, etc. Delivering different liquids at different pressures, temperatures, etc. may, for example, ensure the delivery of proper volumes of the liquids, improve the effectiveness of the delivery, etc. Different liquids delivered using the injection stations described herein may also have different properties such as, for example, viscosity, density, different carrier liquids (e.g., oils versus water-based carriers), etc. Those different properties may or may not result in liquids that are not suitable for mixing prior to delivery to a bird and which, as a result, our preferably delivered using a dual port needle carrier such as that depicted in, e.g., FIGS. 20-22.

Providing two different pump assemblies on a pump base 160 as depicted in connection with the illustrative embodiment of poultry processing system of FIGS. 23-24 may provide the ability to deliver different liquids having different properties and or needing delivery under different conditions. Although the depicted embodiments of pump assemblies are in the form of a peristaltic pump assembly 170 and a piston pump assembly 180, one or more embodiments of poultry processing systems as described herein including injection stations requiring pumps may include any suitable pump assemblies in place of the depicted piston pump assembly 180 and peristaltic pump assembly 170. One or more alternatives for delivering fluids to injection needles and injection stations of poultry processing systems as described herein may include, for example, diaphragm pumps, pressurized bladders, etc.

The depicted illustrative embodiment of the poultry processing system seen in FIGS. 23-24 includes space for up to four different injection stations on the system platform 100 to with each injection station being attached to the system platform 102 at a mounting site 109 on the system platform 102. FIG. 24, in particular, depicts three additional mounting sites 109 on the system platform 102 noting that the right most mounting site 109 is occupied by injection station 110-1 in FIGS. 4-5.

With space for up to four different injection stations on the system platform 102 each of the pump assemblies has the ability to deliver fluids to each of the four different injection stations. In particular, peristaltic pump assembly 170 includes four outlets 172, each of which may be fluidly connected to one of the injection stations mounted on the system platform 102. Similarly, the piston pump assembly 180 includes a set of four piston pumps 184, each of which includes an outlet 182 that may be fluidly connected to one of the injection stations mounted on the system platform 102.

Although the depicted illustrative embodiment of the poultry processing system seen in FIGS. 23-24 includes two different pump assemblies, in one or more alternative embodiments the poultry processing system may include two of the same pump assemblies, i.e., a pair of peristaltic pump assemblies or a pair of piston pump assemblies. Further, although the peristaltic pump assembly 170 is located on the right side of the pumping base 160 while the piston pump assembly 180 is located on the left side of the pumping base 160 as seen in FIG. 24, the positions of the pump assemblies may be swapped such that the peristaltic pump assembly 170 is located on the left side of the pumping base 160 and the piston pump assembly 180 is located on the right side of the pumping base 160.

In one or more embodiments of the poultry processing systems described herein in which pump assemblies are provided to deliver fluids using, e.g., injection stations, the pumping base 160 may be configured to receive a variety of different pump assemblies and to preferably adapt to those different pump assemblies quickly and accurately.

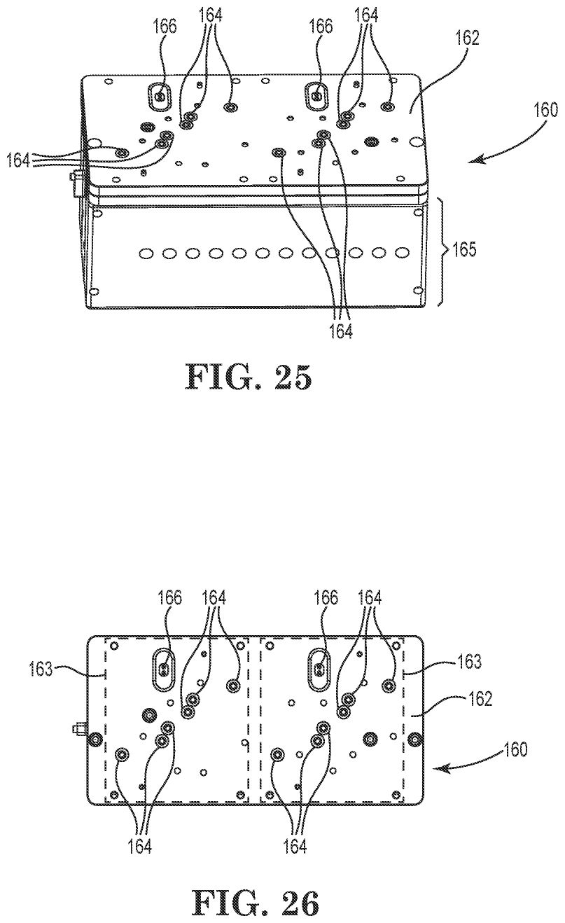

The depicted pumping base 160 is adapted to deliver pneumatic fluid (e.g., air or any other suitable gas) to power the pump assemblies located on the pumping base 160. With reference to FIGS. 25-26, the pumping base 160 includes a pump assembly surface 162 located on or above a manifold 165 configured to deliver pneumatic fluid to a plurality of actuation fluid delivery ports 164 opening on the pump assembly surface 162. Pneumatic fluid may be delivered to the manifold 165 through, e.g., a pneumatic connection that is not depicted.

The depicted illustrative embodiment of pumping base 160 includes a pair of mounts 163 on the pump assembly surface 162 wherein each of the mounts 163 includes a mount perimeter defined by broken lines as depicted in FIG. 26. Within the perimeter of each of the mounts 163, the actuation fluid delivery ports 164 define a delivery port pattern. To facilitate acceptance of a variety of different pump assemblies, each of the mounts 163 includes a set of actuation fluid delivery ports 164 that define the same delivery port pattern in the respective mount perimeters. As a result, different pump assemblies requiring different actuation fluid delivery ports 164 may be swapped on each of the mounts 163 as described herein.

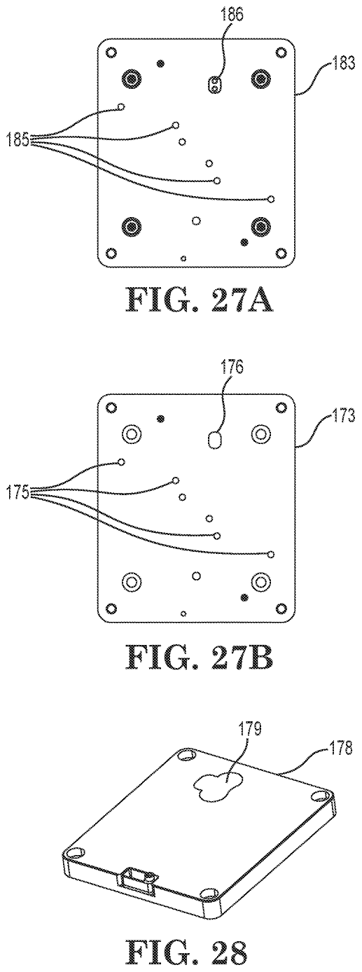

FIG. 27A depicts the bottom surface of the piston pump assembly 180 while FIG. 27B depicts the bottom surface of the peristaltic pump assembly 170. The bottom surface of piston pump assembly 180 is formed on base 183 as depicted in FIGS. 23 and 24 while the bottom surface of the peristaltic pump assembly 170 is formed on base 173 as depicted in FIGS. 23 and 24. Base 183 of piston pump assembly 180 includes a set of piston pump ports 185 providing passage for pneumatic fluid delivered from the pumping base 160 to operate piston pumps 184 of piston pump assembly 180.

Base 183 of piston pump assembly 180 as depicted in FIG. 27A also includes a component 186 of a pump identification system. Identification system component 186 is positioned such that it aligns with a complementary identification system component 166 in the pump mounts 163 on pumping base 160 when the base 183 of piston pump assembly 180 is aligned on either of the pump mounts 163 such that piston pump ports 185 in base 183 are aligned with a selected set of the actuation fluid delivery ports 164 within the pump mounts 163 on pumping base 160.

Similarly, base 173 of peristaltic pump assembly 170 as depicted in FIG. 27B also includes an identification system component 176 located in the base 173. Identification system component 176 is positioned such that it aligns with a complementary identification system component 166 in the pump mounts 163 on pumping base 160 when the base 173 of peristaltic pump assembly 170 is aligned on either of the pump mounts 163 such that the peristaltic pump ports 175 in base 173 are aligned with a selected set of the actuation fluid delivery ports 164 within the pump mounts 163 on pumping base 160.

FIG. 28 depicts a blanking plate 178 that may be positioned on a pump mount 163 when the poultry processing system requires only one pump assembly. The blanking plate 178 includes, in one or more embodiments, an identification system component 179 positioned on the blanking plate 178 such that it is aligned with a complementary identification system component 166 in the pump mounts 163 on pumping base 160 when the blanking plate 178 is aligned on either of the pump mounts 163.

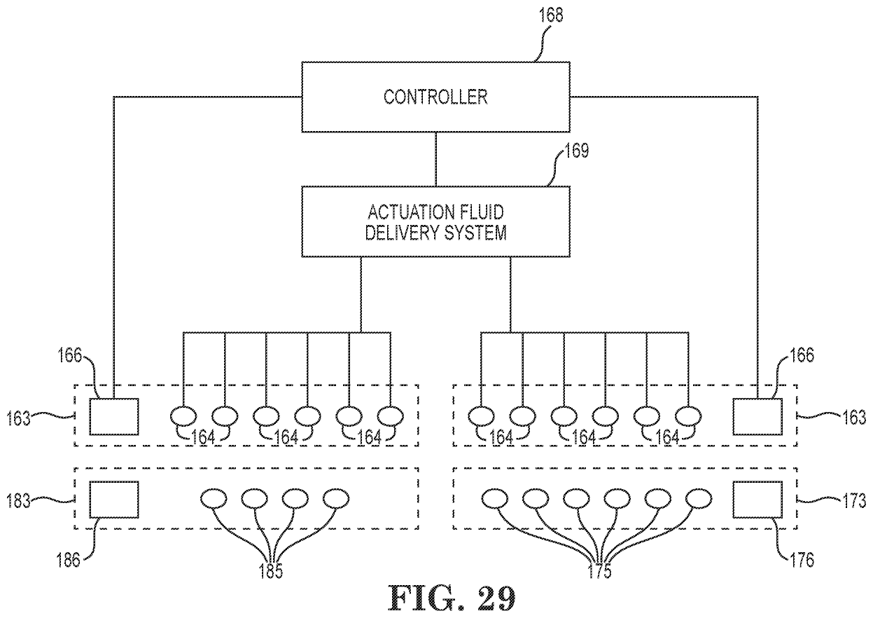

FIG. 29 is a schematic block diagram of a control system that may be used in connection with the pumping base of a poultry processing system as described herein to control the delivery of fluid using pump assemblies mounted on the pumping base. The system may include a controller 168 operably connected to an actuation fluid delivery system 169. The controller 168 may be configured to receive signals from the identification system components and operate the actuation fluid delivery system to deliver actuation fluid to selected delivery ports 164 on the pumping base 160. In particular, the controller 168 may be configured to receive a signal from a first identification component associated with the left side pump mount 163 on pumping base 160 and, based on that signal, deliver actuation fluid to a selected set of delivery ports 164 located in the perimeter of the left side pump mount 163. The controller 168 may also be configured to receive a signal from a second identification component associated with the right side pump mount 163 on pumping base 160 and, based on that signal, deliver actuation fluid to a selected set of delivery ports 164 located in the perimeter of the right side pump mount 163.

The signals provided by the identification components associated with the different pump mounts and/or pump assemblies may, in one or more embodiments, result in delivery of actuation fluid to different sets of delivery ports 164 in the perimeters of the different pump mounts 163 based on the requirements or needs of the type of pump assembly positioned on the pump mount of the pumping base 160. For example, a selected set of four delivery ports 164 in each of the pump mounts 163 may be used to deliver actuation fluid to the ports 185 on the base 183 of a piston pump assembly as depicted in FIGS. 29 and 27A, while a selected set of two delivery ports 164 in each of the pump mounts 163 may be used to deliver actuation fluid to the ports 175 on the base 173 of a peristaltic pump assembly as depicted in FIGS. 29 and 27B.

In one or more embodiments of the poultry processing systems described herein including pump identification systems, the pump identification systems may include a reader component and an identifier component. In one or more embodiments, the identifier component may be attached to the pump assembly while the reader component may be attached to the pumping base. In one or more alternative embodiments, the identifier component may be attached to the pumping base while the reader component is attached to the pump assembly. Regardless of the location of the different components of the pump identification systems, the pump identification systems are configured to deliver a signal to the controller 168 indicative of the type of pump assembly located on a selected pump mount such that the controller can operate the actuation fluid delivery system 169 to deliver actuation fluid to a selected set of delivery ports 164 of the selected pump mount.

The components of the pump identification systems used in one or more embodiments of poultry processing systems described herein may take any suitable form. In one or more embodiments, the components associated with the pump assemblies may take the form of EEPROM units with complementary components located on the pumping base to receive information from the EEPROM units and provide a suitable signal to the controller 168. Other potentially suitable alternative embodiments of pump identification systems may use barcodes, QR codes, RFID tags/devices, Near Field Communication (NFC) tags/devices, or any other suitable technology capable of identifying the type of pump assembly located on the pumping base and provide a signal to the controller for control of the actuation fluid delivery system as needed.

The controller 168 used in one or more embodiments of the poultry processing systems as described herein may be provided in any suitable form and may, for example, include memory and a controller. The controller may, for example, be in the form of one or more microprocessors, Field-Programmable Gate Arrays (FPGA), Digital Signal Processors (DSP), microcontrollers, Application Specific Integrated Circuit (ASIC) state machines, etc. The controller may include one or more of any suitable input devices configured to allow a user to operate the apparatus (e.g., keyboards, touchscreens, mice, trackballs, etc.), as well as display devices configured to convey information to a user (e.g., monitors (which may or may not be touchscreens), indicator lights, etc.).

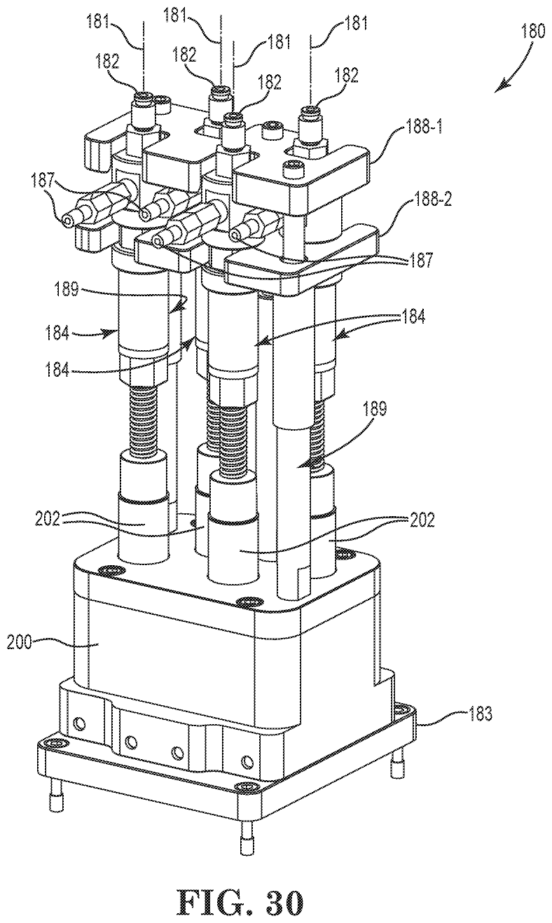

One illustrative embodiment of a pump assembly including a set of piston pumps that may be used in one or more embodiments of a poultry processing system as described herein is depicted in FIG. 30. The pump assembly 180 includes a set of piston pumps 184, each of the piston pumps 184 including an outlet 182 and an inlet 187, with fluid to be pumped entering the piston pumps 184 through their respective inlets 187 and being ejected from the piston pumps 184 through their respective outlets 182. As discussed herein, the outlets 182 of the piston pumps may be attached to one or more injection stations provided in a poultry processing system as described herein such that fluid entering the piston pumps through inlets 187 can be delivered to injection needles of the injection stations of the poultry processing systems through outlets 182.

Each of the piston pumps 184 of pump assembly 180 is aligned along a piston axis 181 above a pump base 200, with each of the piston pumps 184 being aligned with a piston driver 202 on the pump base 200. The piston pumps 184 are retained in position above the pump base 200 and piston drivers 202 by a clamping apparatus that includes a fixed cradle 188-1 and a movable cradle 188-2, with the fixed cradle 188-1 being supported at a fixed location above the pump base 200 while the movable cradle 188-2 is urged in the direction of the fixed cradle 188-1 by biasing members 189 (which may be in the form of, e.g., compression springs or any other suitable biasing apparatus such as, e.g., resilient elastomeric members, compressible bladders, etc.). As a result, a portion of the piston pumps 184 is clamped between the fixed cradle 188-1 and the movable cradle 188-2 to retain the piston pumps properly positioned above piston drivers 202 of pump base 200.

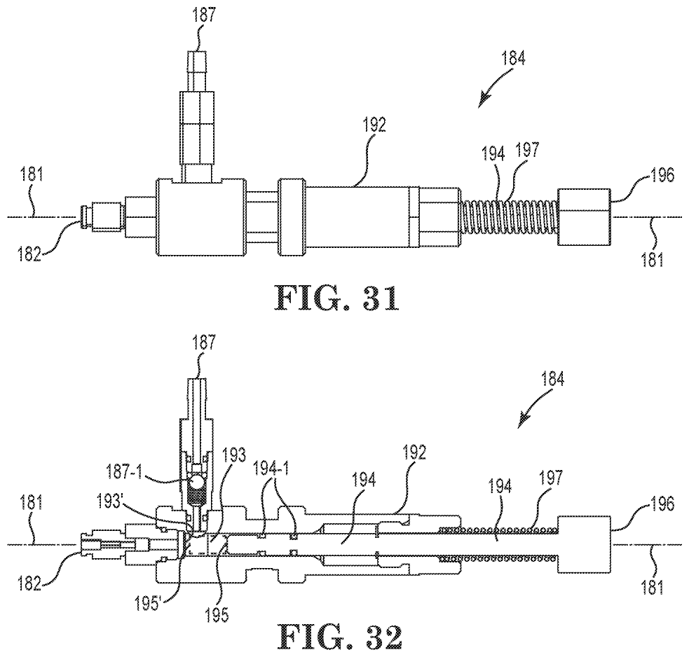

One illustrative embodiment of a piston pump that may be used in the piston pump assemblies of poultry processing systems as described herein is depicted in FIGS. 31-32. The depicted piston pump 184 includes a housing 192 defining a piston chamber 193 along with a chamber inlet 187 in fluid communication with the piston chamber 193 and a chamber outlet 182 also in fluid communication with the piston chamber 193.

In one or more embodiments, a flow control apparatus 187-1 is provided to prevent fluid from leaving the piston chamber 193 through the chamber inlet 187. In the depicted embodiment, the flow control apparatus 187-1 is in the form of a check valve and, more specifically, in the form of a ball valve although any suitable check valve or other one-way fluid control device could be substituted for the depicted ball valve, e.g., a diaphragm check valve, swing/tilting disc check valve, clapper valve, duck bill valve, etc.

A piston 194 is configured for movement within the piston chamber along a piston axis 181. The piston 194 includes a forward end 195 located within the piston chamber 193 and a trailing end 196 located outside of the piston chamber 193. Movement of the forward end 195 of the piston 194 along the piston axis 181 within the piston chamber 193 towards the chamber outlet 182 forces fluid in the piston chamber 193 out of the piston chamber 193 through the chamber outlet 182. In one or more embodiments, a flow control device may be provided between the piston chamber 193 and the chamber outlet 182 to provide additional control over fluid flow into and/or out of the piston chamber 193.

In the depicted illustrative embodiment, the chamber outlet 182 is aligned with the piston axis 181 with that alignment potentially providing improvements in fluid flow out of the chamber outlet 182. In one or more embodiments, providing a relatively straight flow of fluid out of the piston pump 184 can reduce turbulence in that flow of fluid. That reduced turbulence may be advantageous when the fluids being delivered using the piston pumps 184 are sensitive to turbulence.

Advancement of the forward end 195 of the piston 194 is depicted in broken lines 195′ in FIG. 32. The amount of fluid delivered through chamber outlet 182 by the piston pump 184 is defined by the difference between the location of the forward end 195 of the piston pump 194 and the location of the forward and of the piston 194 as depicted by broken lines 195′.

The depicted piston pump 184 also includes a biasing apparatus 197 provided to move the piston 194 to a rest position. The piston 194 of the depicted piston pump 184 is in its rest position as depicted in FIGS. 31-32, with the rest position of the piston 194 providing a resting volume in the piston chamber 193. The resting volume is defined by a resting distance measured between the forward end 195 of the piston 194 within the piston chamber 193 and a terminal stop 193′ located in the piston chamber 193 when the piston 194 is in the rest position as seen in FIG. 32. The terminal stop 193′ is the location past which the terminal end 195 of the piston 194 cannot advance due to mechanical interference with structures inside the piston chamber 193. In other words, the piston 194 would bottom out, i.e., would no longer advance, within the piston chamber 193 at the terminal stop 193′.

The biasing apparatus 197 depicted in connection with piston pump 184 is in the form of a coil spring although any suitable biasing apparatus could be used to move the piston 194 to its rest position in a piston pump as described herein. Examples of other potentially suitable biasing apparatus include, but are not limited to, resilient elastomeric members, pressurize bladders, pneumatic cylinders, hydraulic cylinders, solenoid devices, magnetic apparatus, etc.

Control over the delivery of accurate dosages of fluids using piston pumps is difficult when that control is provided solely by attempts to control the travel distance of pistons within the piston pumps accurately between piston pump actuation cycles, particularly when changes in the volume of fluid delivered by the piston pumps are required. In the poultry processing systems described herein, dosage pucks may be used to provide precise control over the amount of fluid delivered using the piston pumps of poultry processing systems described herein by removing piston travel control to a location outside of the piston pumps. In one or more embodiments, the dosage pucks have selected heights that correspond to a selected travel distance that, in turn, correspond to selected volumes within the piston chamber such that the stroke distance of the piston within the piston pump can be controlled precisely using dosage pucks having known heights.

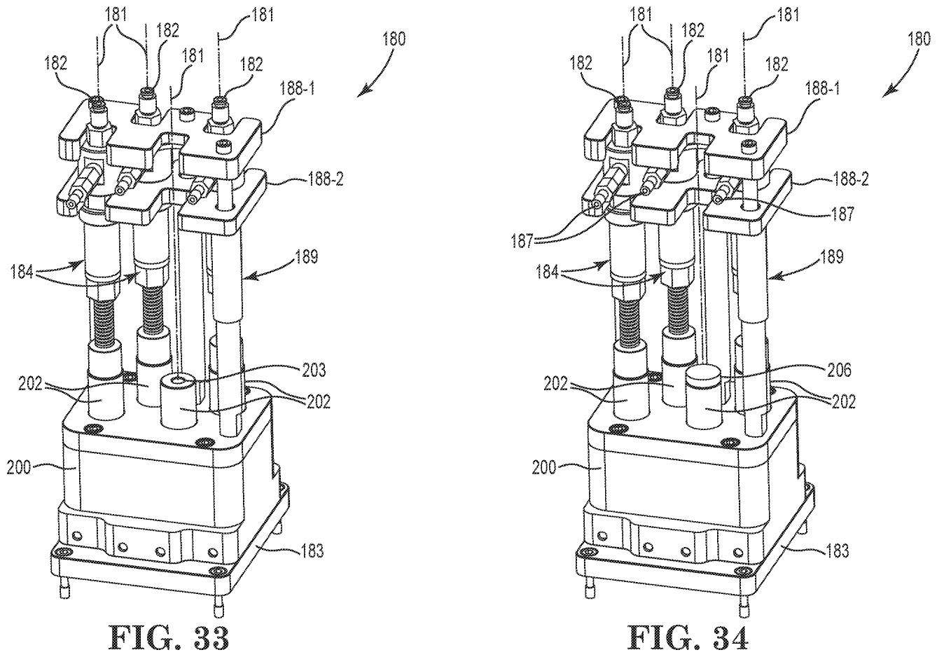

The piston pump assembly 180 of FIG. 30 is depicted in FIGS. 33-34 with one of the piston pumps 184 removed. The missing piston pump 184 exposes the piston driver 202 of the pump base 200. When the piston pumps 184 are located within the piston pump assembly 180 the piston drivers 202 are proximate the trailing ends 196 of the pistons 194 in the piston pumps 184 (see, e.g., FIGS. 31-32) when the piston driver 202 is in the advanced position as seen in FIGS. 33-34 and the piston 194 is in its rest position as also seen in FIGS. 33-34 as well as FIGS. 31-32.

Removal of the piston pump 184 from one of the piston drivers 202 exposes a cavity 203 in the piston driver 202. As will be described herein, the cavity 203 and piston driver 202 can be used to secure a dosage puck 206 on the piston driver 202. The addition of the dosage puck 206 on piston driver 202 raises the height of the piston driver such that the terminal end 196 of a piston pump 184 positioned on the dosage puck 206 is located closer to the chamber outlet 182 when the piston driver 202 is in its advanced position to provide dosage control using the piston pumps as described herein.

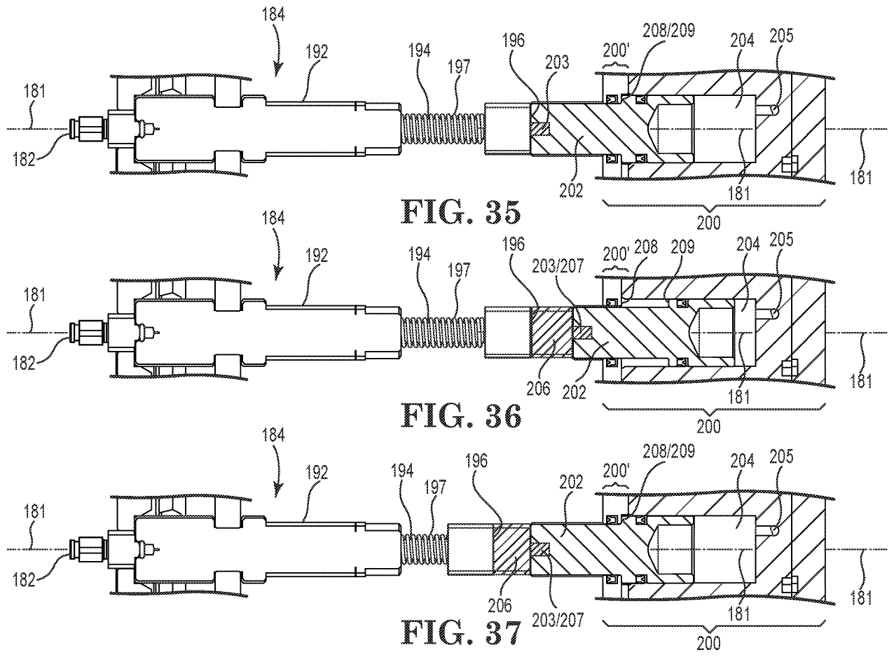

FIGS. 35-37 depict the operation and effect of dosage pucks on the piston pumps in pump assemblies of multiprocessing systems as described herein. In FIGS. 35-37, the pump base 200 is depicted in a partial cross-sectional view with the piston driver 202 and dosage puck 206 (when present) also depicted in cross-section.

FIG. 35 depicts the piston pump 184 located on piston driver 202 with the piston 194 in its rest position as discussed in connection with the piston pump 184 as depicted in FIGS. 31-32. In the rest position the trailing end 196 of the piston 194 is located proximate the piston driver 202. Also depicted in FIG. 35 is the cavity 203 in the piston driver 202. The pump base 200 as depicted in FIG. 35 includes a cavity 204 in which piston driver 202 moves when moving from its advanced position as seen in FIG. 35 (noting that the piston drivers 202 of pump base 200 are also in the advanced positions as depicted in FIGS. 30 and 33-34).

The depicted illustrative embodiment of piston driver 202 includes a shoulder 209 which, when the piston driver 202 is in its advanced position as seen in FIG. 35 is in an abutting relationship with stop 208 provided in piston driver cavity 204. In the depicted embodiment of pump base 200 the stop 208 is provided in a cover plate 200′ that faces the piston pump 184 although this construction is not required.

The depicted pump base 200 includes features configured to provide for pneumatic driving of the piston driver 202. In particular, the piston driver cavity 204 is fed by an inlet 205 which delivers air or any other suitable gas into the piston driver cavity 204 to move the piston driver 202 to its advanced position as depicted in FIG. 35 from a rest position as seen in FIG. 36.

With reference to FIG. 36, a dosage puck 206 is located in between the piston driver 202 and the trailing end 196 of the piston 194 of the piston pump 184. The dosage puck 206 has a selected height as measured along the piston axis 181 that corresponds to a selected volume within the piston chamber 193 of the piston pump 184 between the forward end 195 of the piston 194 and the chamber outlet 182 such that movement of the piston 194 towards the chamber outlet 182 over a stroke distance equal to the height of the dosage puck 206 moves the selected volume of fluid out of the piston chamber 193 through the chamber outlet 182.

The depicted dosage puck 206 includes a stem 207 configured to be received in the cavity 203 provided in the piston driver 202, noting that the cavity 203 is seen in FIG. 33. Providing dosage pucks 206 with stems 207 configured to be located within cavities 203 in piston drivers 202 ensures that the dosage pucks 206 remain properly positioned between the piston driver 202 and the trailing end 196 of a piston pump. As depicted in FIG. 36, the shoulder 209 on the piston driver 202 is spaced apart from the stop 208 provided in the piston driver cavity 204. The dosage puck 206 and piston driver 202 are displaced rightward as seen in FIG. 36 by the biasing apparatus 197 of piston pump 184. Free movement of the piston driver 202 in the piston driver cavity 204 is, in one or more embodiments such as that depicted in FIG. 36, restricted by the friction between the seals on the piston driver 202 and the interior surface of the piston driver cavity 204.

With reference to FIG. 37, the piston actuator is operated to bias or force the piston driver 202 towards the advanced position with a pumping force greater than the biasing force provided by the biasing apparatus 197 of the piston pump 184. The pumping force provided by the piston actuator is large enough such that the piston driver 202 moves towards the advanced position and the piston 194 is moved towards the chamber outlet over the stroke distance when the dosage puck 206 is located between the piston driver 202 and a trailing end 196 of the piston 194. That stroke distance is, in the depicted embodiment, equal to the height of the dosage puck 206 as measured along the piston axis 181.

Because the dosage puck 206 moves the shoulder 209 of the piston driver 202 away from the stop 208 in piston driver cavity 204 by a distance equal to the height of the dosage puck 206, operation of the piston actuator to move the piston driver 202 and the dosage puck 206 towards the piston pump 184 moves the piston 194 over a distance equal to, but no more than, the height of the dosage puck 206 as measured along the piston axis 181. As a result, the height of the dosage puck 206 sets the stroke volume of the piston pump 184 and, therefore, also sets the volume of fluid delivered by the piston pump 184 during actuation of the piston driver 202.

More importantly, the combination of dosage pucks and piston drivers that have a fixed or known advanced position provided by the stop 208 and shoulder 209 described herein provides accurate and repeatable control over the stroke distance of pistons in piston pumps as described herein. That accurate and repeatable control over the stroke distance provides, in turn, accurate and repeatable control over the volumes of fluid delivered using the piston pumps described herein because the stroke distance is fixed based on the height of the dosage pucks.

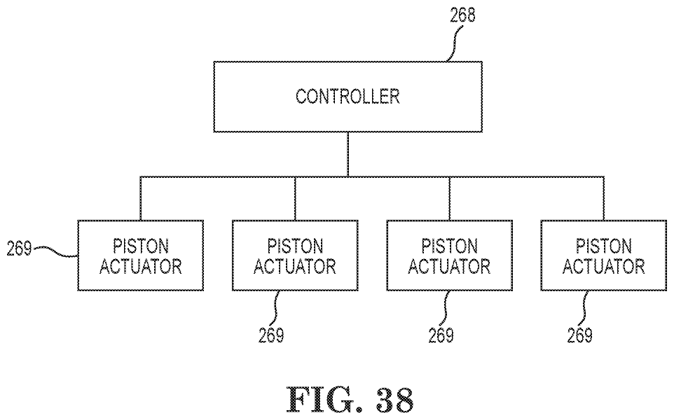

One illustrative embodiment of a control system and set of piston actuators that may be used with one or more embodiments of piston pump assemblies of poultry processing systems as described herein is depicted in the schematic block diagram of FIG. 38. The control system includes a controller 268 operably connected to each piston actuator 269. In the depicted embodiment a set of four piston actuators 269 are depicted because the illustrative embodiment of piston assembly 180 includes four piston pumps. Providing piston actuators for each of the piston pumps provides control over actuation of each piston pump individually which, although may be preferred, is not required for proper operation of the poultry processing systems as described herein.

As discussed herein, the piston actuators 269 may be in the form of fluid actuators in which pressurized fluid is delivered to a piston driver chamber 204. In one or more embodiments, the pressurized fluid may be in the form of air such that the piston actuators are pneumatic actuators, but it should be understood that any suitable alternative mechanisms or techniques may be used to drive the piston drivers including, but not limited to, solenoids, hydraulic pressure, cams driven by rotating motors (electric, hydraulic, pneumatic, etc.), etc.

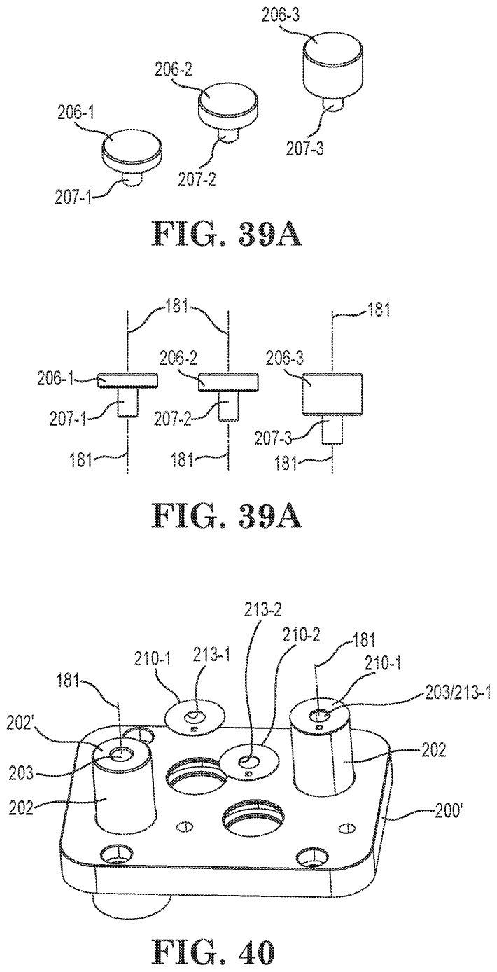

FIGS. 39A-39B depict sets of dosage pucks 206 that may be provided for use in a piston pump assembly of a poultry processing system as described herein. By way of example only, the dosage puck 206-1 may have a height as measured along a representative piston axis 181 that is the equivalent of a first selected volume of fluid to be delivered by a piston pump as described herein. Dosage puck 206-2 has a second height as measured along a representative piston axis 181 that is the equivalent of a second selected volume of fluid to be delivered by a piston pump as described herein. Dosage puck 206-3 has a third height as measured along a representative piston axis 181 that is the equivalent of a third selected volume of fluid to be delivered by piston pump as described herein. The dosage puck 206-1, having the shortest height, delivers the least amount of fluid while the dosage puck 206-3 having the largest height delivers the greatest amount of fluid, with the dosage puck 206-2 having an intermediate height delivering an intermediate volume of fluid.

Each of the dosage pucks 206-1, 206-2, and 206-3 preferably includes a stem 207-1, 207-2 and 207-3 (respectively) configured to be inserted into the cavity 203 of a piston driver 202 as described herein to assist in retaining the dosage pucks on a piston driver 202.

While dosage pucks alone can be used to control the volume of fluid delivered using piston pumps as described herein, FIG. 40 depicts shims that may be used in conjunction with the dosage pucks to adjust the volume of fluid delivered by the piston pumps and or to facilitate proper operation of the dosage pucks in situations in which the trailing and 196 of a piston 194 of a piston pump 184 as described herein does not contact the piston driver 202 when the piston driver 202 is in its advanced position.

In particular, the cover plate 200′ and a pair of piston drivers 202 are depicted in FIG. 40 along with a set of shims 210-1 and 210-2. With reference to the piston driver 202 on the left side of the assembly depicted in FIG. 40, the piston driver 202 includes a cavity 203 that extends into the piston driver 202 from its upper surface 202′. The piston driver 202 on the right side of the assembly depicted in FIG. 40 includes a piston driver having a cavity 203 with a shim 210-1 located on the upper surface 202′ of the piston driver 202. The shim 210-1 includes an aperture 213-1 that corresponds to the cavity 203 in the piston driver 202 such that insertion of a stem of a dosage puck into the cavity 203 is allowed.

Because the purpose of the shims 210-1 and 210-2 is to provide finer adjustments in the volume of fluid delivered by the piston pumps of poultry processing systems described herein and/or to provide proper spacing between the piston driver's and trailing ends of pistons as described herein, the height of the shims as measured along the piston axes 181 is typically significantly smaller than the height of the dosage pucks.

By way of example only, dosage puck 206-1 may have a height that, when used with a piston pump 184 as described herein, provides a volume of fluid equal to 0.15 mL, dosage puck 206-2 may have a height that, when used with a piston pump 184 as described herein, provides a volume of fluid equal to 0.2 mL, and dosage puck 206-3 may have a height that, when used with a piston pump 184 as described herein, provides a volume of fluid equal to 0.5 mL. In contrast, shim 210-1 may have a height that, when used with a piston pump 184 as described herein, provides a volume of fluid equal to 0.005 mL while shim 210-2 may have a height that when used with a piston pump 184 as described herein, provides a volume of fluid equal to 0.010 mL. As a result, shims can be used to provide volumes of fluid in between those provided by the different dosage pucks.

As discussed herein, it may be preferred that, when the piston driver 202 is in its advanced position and the piston 194 of the piston pumps 184 is in its rest position, the piston driver 202 contacts the trailing end 196 of the piston 194 such that no gap is present between the piston driver 202 and the trailing end 196 of the piston 194. Due to manufacturing tolerances (and possibly other factors) such tight control may not be achievable in all instances. In fact, in some instances it may be preferred that the piston driver 202 does not contact the trailing end 196 of a piston 194 such that a gap is present between the piston driver 202 and the trailing end 196 of the piston 194.

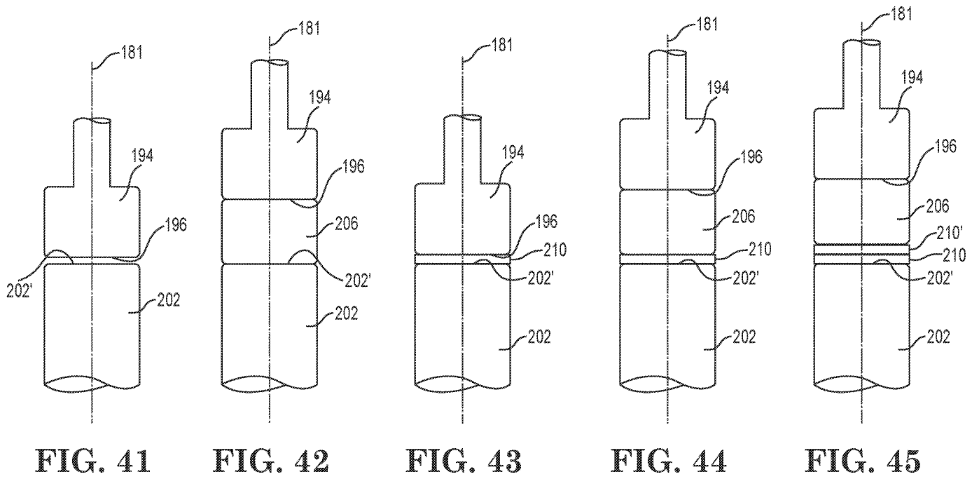

Such a situation is depicted in FIG. 41 where surface 202′ of piston driver 202 does not contact the trailing end 196 of piston 194 (noting that piston axis 181 is provided for reference).

With reference to FIG. 42, a dosage puck 206 is positioned between the surface 202′ of piston driver 202 and the trailing end 196 of piston 194 as described herein. Although dosage puck 206 contacts both the surface 202′ of piston driver 202 and the trailing end 196 of piston 194, movement of the piston driver 202 and dosage puck 206 towards the piston 194 would not result in movement of the piston 194 over the full height of the dosage puck 206 as measured along piston axis 181. In particular, piston 194 would move over a distance equal to the height of the dosage puck 206 less the height of the gap between surface 202′ of piston driver 202 and trailing and 196 of piston 194 as depicted in FIG. 41.

To address that issue, a shim 210 may be provided between the dosage puck 206 and the surface 202′ of piston driver 202 to occupy the height of the gap between surface 202′ of piston driver 202 and trailing end 196 of piston 194. With reference to FIG. 43, a shim 210 is located between the surface 202′ of piston driver 202 and the trailing end 196 of piston 194. The height of the shim 210 is preferably at least as great as the height of the gap between the surface 202′ and trailing and 196 as depicted in FIG. 41.

FIG. 44 depicts the shim 210 located between dosage puck 206 and piston driver 202 with the dosage puck 206 contacting the trailing end 196 of piston 194. As a result, movement of the piston driver 202 to its actuation position as seen in, e.g., FIG. 37 results in movement of the piston 194 over the full height of the dosage puck 206.

FIG. 45 depicts yet another variation in which a pair of shims 210 and 210′ are provided between the surface 202′ of piston driver 202 and the trailing end 196 of the piston 194. In such an arrangement, shim 210 may be provided to occupy a gap as depicted in FIG. 41 while shim 210′ is provided to increase the dosage delivered during operation of the piston pump by increasing the travel distance of the piston 194 by a distance equal to the height of shim 210′.

In one or more embodiments of a piston pump used in a poultry processing system as described herein, the piston pump may (with reference to, e.g., FIG. 32) define a resting volume defined by a resting distance measured between the forward end 195 of the piston 194 and a terminal stop 193′ within the piston chamber 193, the terminal stop 193′ located between the chamber outlet 182 and the forward end 195 when the piston 194 is in the rest position. In one or more embodiments, the height of any dosage puck used in connection with the piston pump is preferably less than or equal to the resting distance to prevent limiting the stroke distance due to interference between the terminal stop 193′ and the forward end 195 of the piston 194.

Although depicted as being located between the dosage puck 206 and the piston driver 202, it will be understood that any shim 210 could be provided on the opposite end of the dosage puck 206 such that shim 210 is located between the trailing end 196 of piston 194 and the dosage puck 206.

### ILLUSTRATIVE EMBODIMENTS

Following are some illustrative embodiments of some apparatus, systems, and methods described herein.

Embodiment 41 is a piston pump assembly comprising: a piston pump comprising: a housing defining a piston chamber, a chamber inlet in fluid communication with the piston chamber, a chamber outlet in fluid communication with the piston chamber, a flow control apparatus configured to prevent fluid from leaving the piston chamber through the chamber inlet, a piston comprising a forward end located in the piston chamber and a trailing end located outside of the piston chamber, the piston configured for movement along a piston axis within the piston chamber, wherein movement of the forward end of the piston along the piston axis within the piston chamber towards the chamber outlet is configured to force fluid in the piston chamber out of the piston chamber through the chamber outlet, and biasing apparatus configured to move the piston to a rest position, wherein the biasing apparatus is configured to resist movement of the piston towards the chamber outlet; a pump actuator comprising a piston driver, wherein the pump actuator is configured to move the piston driver towards the chamber outlet to an advanced position, wherein the piston driver is proximate the trailing end of the piston when the piston driver is in the advanced position and the piston is in the rest position; and a dosage puck configured for placement between the trailing end of the piston and the piston driver, wherein the dosage puck comprises a selected height that corresponds a selected volume within the piston chamber between the forward end of the piston and the chamber outlet such that movement of the piston towards the chamber outlet over a stroke distance equal to the selected height of the dosage puck moves the selected volume of fluid out of the piston chamber through the chamber outlet.

Embodiment 42 is a piston pump assembly according to Embodiment 41, wherein the piston driver is spaced away from the advanced position when the dosage puck is located between the piston driver and the trailing end of the piston, and wherein the assembly comprises a controller operably connected to the piston actuator, wherein the controller is configured to selectively operate the piston actuator to bias the piston driver to the advanced position such that the piston is moved towards the chamber outlet over the stroke distance when the dosage puck is located between the piston driver and the trailing end of the piston.

Embodiment 43 is a piston pump assembly according to any one of Embodiments 41 to 42, wherein, when the piston driver is in the advanced position and the piston is in the rest position, the piston driver does not contact the trailing end of the piston such that a gap is present between the piston driver and the trailing end of the piston.

Embodiment 44 is a piston pump assembly according to Embodiment 43, wherein the assembly comprises a shim located between the piston driver and the trailing end of the piston, wherein the shim occupies the gap between the piston driver and the trailing end of the piston.

Embodiment 45 is a piston pump according to any one of Embodiments 41 to 44, wherein the piston pump comprises a resting volume defined by a resting distance measured between the forward end of the piston and a terminal stop within the piston chamber, the terminal stop located between the chamber outlet and the forward end when the piston is in the rest position, wherein the selected height of the dosage puck is less than or equal to the resting distance.

Embodiment 46 is a piston pump according to any one of Embodiments 41 to 44, wherein the piston pump comprises a resting volume defined by a resting distance measured between the forward end of the piston and a terminal stop within the piston chamber, the terminal stop located between the chamber outlet and the forward end when the piston is in the rest position, wherein the selected height of the dosage puck is less than the resting distance.

Embodiment 47 is a piston pump assembly according to any one of Embodiments 41 to 46, wherein the piston actuator comprises a fluid pressure actuator.

Embodiment 48 is a piston pump assembly according to any one of Embodiments 41 to 47, wherein the flow control apparatus comprises a check valve.

Embodiment 49 is a piston pump assembly according to any one of Embodiments 41 to 48, wherein the biasing apparatus comprises a spring.

Embodiment 50 is a piston pump assembly according to any one of Embodiments 41 to 49, wherein the chamber outlet is aligned with the piston axis.

Embodiment 51 is a method of adjusting the pumping volume of a piston pump comprising a piston having a piston chamber a chamber outlet in fluid communication with the piston chamber, a piston having a forward end located in the piston chamber and a trailing end located outside of the piston chamber, the method comprising: biasing the piston away from the chamber outlet to a rest position in the piston chamber; pumping a selected volume of fluid out of the piston chamber by advancing the piston through the piston chamber away from the rest position and towards the chamber outlet using a piston driver acting on the trailing end of the piston; and adjusting the selected volume by positioning a selected dosage puck between the trailing end of the piston and a piston driver, wherein the selected dosage puck comprises a selected height that corresponds to the selected volume within the piston chamber between the forward end of the piston and the chamber outlet such that movement of the piston towards the chamber outlet over a stroke distance equal to the selected height of the dosage puck moves the selected volume of fluid out of the piston chamber through the chamber outlet.

Embodiment 52 is a method according to Embodiment 51, wherein the method comprises biasing the piston driver towards a fixed advanced position with a base force such that the piston driver exerts a driving force equal to or less than the base force on the trailing end of the piston when the dosage puck is located between the piston driver and the trailing end of the piston.

Embodiment 53 is a method according to any one of Embodiments 51 to 52, wherein the method comprises, before positioning a selected dosage puck between the trailing end of the piston and a piston driver, determining if a gap exists between the piston driver and the trailing end of the piston when the piston is in the rest position and the piston driver is in the advanced position.

Embodiment 54 is a method according to Embodiment 53, the method comprising positioning a shim in the gap between the piston driver and the trailing end of the piston.

Embodiment 55 is a method according to any one of Embodiments 51 to 54, wherein the piston pump comprises a resting volume defined by a resting distance measured between the forward end of the piston within the piston chamber when the piston is in the rest position, wherein the selected height of the dosage puck is less than or equal to the resting distance.

Embodiment 56 is a method according to any one of Embodiments 51 to 54, wherein the piston pump comprises a resting volume defined by a resting distance measured between the forward end of the piston within the piston chamber when the piston is in the rest position, wherein the selected height of the dosage puck is less than the resting distance.

Embodiment 61 is a poultry processing system comprising:
a system platform and a support ring mounted on a base, wherein the system platform comprises an injection sector comprising a plurality of injection sites, wherein one or both of the system platform and the support ring are configured to rotate relative to each other about a platform axis extending through the system platform, the support ring, and the base;
a plurality of bird supports mounted on a support ring outside of a perimeter of the system platform, wherein each bird support of the plurality of bird supports is configured to retain a bird in a selected orientation relative to the platform axis at each injection site of the plurality of injection sites, wherein rotation of one or both of the system platform and the support ring about the platform axis moves the plurality of bird supports into and out of alignment with the plurality of injection sites;
an injection station attached to the system platform at each injection site of the plurality of injection sites, wherein each injection station comprises a needle assembly comprising an injection needle, a needle guide, and a needle actuator operably connected to the injection needle, wherein the needle actuator is configured to move the injection needle between an injection position and a retracted position along an injection axis, wherein a distal end of the injection needle extends out of a delivery site of the needle guide along the injection axis when the injection needle is in the injection position;
a first pump positioned on a first mount of a pumping base attached to the system platform, wherein the first mount comprises a first mount perimeter and a first plurality of actuation fluid delivery ports located within the first mount perimeter, wherein the first plurality of actuation fluid delivery ports defines a first delivery port pattern in the first mount perimeter;
a second pump positioned on a second mount of the pumping base attached to the system platform, wherein the second mount comprises a second mount perimeter and a second plurality of actuation fluid delivery ports located within the second mount perimeter, wherein the second plurality of actuation fluid delivery ports defines a second delivery port pattern in the second mount perimeter, wherein the second delivery port pattern is the same as the first delivery port pattern;
an actuation fluid delivery system configured to deliver actuation fluid to the first plurality of actuation fluid delivery ports and the second plurality of actuation fluid delivery ports;
a first pump identification system configured to provide a first signal identifying the first pump;
a second pump identification system configured to provide a second signal identifying the second pump;
a controller operably connected to the actuation fluid delivery system, the first pump identification system, and the second pump identification system, wherein the controller is configured to:
   receive the first signal and the second signal;
   operate the actuation fluid delivery system to deliver actuation fluid to selected delivery ports of the first plurality of actuation fluid delivery ports at selected times based at least in part on the first signal; and
   operate the actuation fluid delivery system to deliver actuation fluid to selected delivery ports of the second plurality of actuation fluid delivery ports at selected times based at least in part on the second signal.

Embodiment 62 is a system according to Embodiment 61, wherein the first pump identification system comprises a first identifier and a first reader, wherein the first reader is configured to read the first identifier and emit a selected first signal indicative of a pump type for the first pump.

Embodiment 63 is a system according to Embodiment 62, wherein the first identifier is attached to the first pump and the first reader is attached to the pumping base; or the first reader is attached to the first pump and the first identifier is attached to the pumping base.

Embodiment 64 is a system according to Embodiment 62, wherein the second pump identification system comprises a second identifier and a second reader, wherein the second reader is configured to read the second identifier and emit a selected second signal indicative of a pump type for the second pump.

Embodiment 65 is a system according to Embodiment 64, wherein the second identifier is attached to the second pump and the second reader is attached to the pumping base; or the second reader is attached to the second pump and the second identifier is attached to the pumping base.

Embodiment 66 is a system according to any one of Embodiments 61 to 66, wherein the pump type is selected from the group of a piston pump and a peristaltic pump.

All references and publications cited herein are expressly incorporated herein by reference in their entirety into this disclosure, except to the extent they may directly contradict this disclosure. Although specific illustrative embodiments have been described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims.

1. A poultry processing system comprising:
a system platform and a support ring mounted on a base, wherein the system platform comprises an injection sector comprising a plurality of mounting sites, wherein one or both of the system platform and the support ring are configured to rotate relative to each other about a platform axis extending through the system platform, the support ring, and the base;
a plurality of bird supports mounted on a support ring outside of a perimeter of the system platform, wherein each bird support of the plurality of bird supports is in a selected orientation relative to the platform axis at each mounting site of the plurality of mounting sites, wherein rotation of one or both of the system platform and the support ring about the platform axis moves the plurality of bird supports into and out of alignment with the plurality of mounting sites, and wherein each bird support of the plurality of bird supports is configured to retain a bird in a selected orientation relative to the platform axis;
an injection station attached to the system platform at a selected mounting site of the plurality of mounting sites, wherein the injection station comprises:
a needle assembly comprising an injection needle, a needle guide, and a needle actuator operably connected to the injection needle, wherein the needle actuator is configured to move the injection needle between an injection position and a retracted position along an injection axis, wherein a distal end of the injection needle extends out of a delivery site of the needle guide along the injection axis when the injection needle is in the injection position, and
an injection carriage operably connected to a carriage actuator, wherein the needle assembly, the needle guide, and the needle actuator are mounted on the injection carriage,
wherein the carriage actuator is configured to rotate the injection carriage about a carriage axis between a standby position and an actuation position,
and wherein the injection axis defines a base approach angle relative to the platform axis when the injection carriage is in the actuation position such that the injection needle approaches a bird support positioned at the selected mounting site at the base approach angle; and
a shim configured to be positioned between the base of the injection station and the selected mounting site, wherein, when positioned between the base of the injection station and the selected mounting site, the shim positions the base above the selected mounting site and rotates the injection axis of the injection station to an adjusted approach angle relative to the platform axis such that the injection needle approaches a bird support positioned at the selected mounting site at the adjusted approach angle.

2. A poultry processing system according to claim 1, wherein the injection axis of the injection station rotates about the delivery site of the needle guide when moving between the base approach angle and the adjusted approach angle such that the delivery site of the needle guide is positioned at the same location relative to the platform axis when the injection axis is in the base approach angle and when the injection axis is in the adjusted approach angle.

3. A poultry processing system according to claim 2, wherein the shim rotates the injection axis about the delivery site 2 degrees or more.

4. A poultry processing system according to claim 1, wherein the injection axis extends through the carriage axis.

5. A poultry processing system according to claim 1, wherein the injection station attached to the system platform at the selected mounting site comprises a first injection station of a plurality of injection station attached to the system platform, wherein the plurality of poultry processing system comprises a second injection station attached to the system platform at a second selected mounting site of the plurality of mounting sites, and wherein the second injection station comprises: a second needle assembly comprising a second injection needle, a second needle guide, and a second needle actuator operably connected to the second injection needle, wherein the second needle actuator is configured to move the second injection needle between an injection position and a retracted position along a second injection axis, wherein a distal end of the second injection needle extends out of a second delivery site of the second needle guide along the second injection axis when the second injection needle is in the second injection position, and a second injection carriage operably connected to a second carriage actuator, wherein the second needle assembly, the second needle guide, and the second needle actuator are mounted on the second injection carriage, wherein the second carriage actuator is configured to rotate the second injection carriage about a second carriage axis between a standby position and an actuation position, and wherein the second injection axis defines a second base approach angle relative to the platform axis when the second injection carriage is in the actuation position such that the second injection needle approaches a bird support positioned at the second selected mounting site at the second base approach angle, wherein the base approach angle of the first injection station and the second base approach angle are the same.

6. A poultry processing system according to claim 5, wherein the poultry processing system comprises a second shim configured to be positioned between the base of the second injection station and the second selected mounting site, wherein, when positioned between the base of the second injection station and the second selected mounting site, the second shim positions the base of the second injection station above the second selected mounting site and rotates the second injection axis of the second injection station to a second adjusted approach angle relative to the platform axis such that the second injection needle approaches a bird support positioned at the second selected mounting site at the second adjusted approach angle.

7. A poultry processing system according to claim 6, wherein the second injection axis of the second injection station rotates about the second delivery site of the second needle guide when moving between the second base approach angle and the second adjusted approach angle such that the second delivery site of the second needle guide is positioned at the same location relative to the platform axis when the second injection axis is in the second base approach angle and when the second injection axis is in the second adjusted approach angle.

8. A poultry processing system according to claim 1, wherein each shim of the plurality of shims positions the base and the injection axis of each injection station of the plurality of injection station at the same selected adjusted angle relative to the platform axis.

9. A poultry processing system according to claim 1, wherein the injection station comprises a carriage damper operably connected to the injection carriage, the carriage damper configured to limit rotational velocity of the injection carriage during rotation of the injection carriage from the standby position to the actuation position.

10-12. (canceled)

13. A poultry processing system according to claim 1, wherein the system comprises a piston pump assembly in fluid communication with the injection needle of the injection station, wherein the piston pump assembly comprises:
a piston pump comprising:
a housing defining a piston chamber,
a chamber inlet in fluid communication with the piston chamber,
a chamber outlet in fluid communication with the piston chamber,
a flow control apparatus configured to prevent fluid from leaving the piston chamber through the chamber inlet,
a piston comprising a forward end located in the piston chamber and a trailing end located outside of the piston chamber, the piston configured for movement along a piston axis within the piston chamber, wherein movement of the forward end of the piston along the piston axis within the piston chamber towards the chamber outlet is configured to force fluid in the piston chamber out of the piston chamber through the chamber outlet, and
biasing apparatus configured to move the piston to a rest position, wherein the biasing apparatus is configured to resist movement of the piston towards the chamber outlet;
a pump actuator comprising a piston driver, wherein the pump actuator is configured to move the piston driver towards the chamber outlet to an advanced position, wherein the piston driver is proximate the trailing end of the piston when the piston driver is in the advanced position and the piston is in the rest position; and
a dosage puck configured for placement between the trailing end of the piston and the piston driver, wherein the dosage puck comprises a selected height that corresponds to a selected volume within the piston chamber between the forward end of the piston and the chamber outlet such that movement of the piston towards the chamber outlet over a stroke distance equal to the selected height of the dosage puck moves the selected volume of fluid out of the piston chamber through the chamber outlet.

14. A poultry processing system according to claim 13, wherein the piston driver is spaced away from the advanced position when the dosage puck is located between the piston driver and the trailing end of the piston, and wherein the assembly comprises a controller operably connected to the piston actuator, wherein the controller is configured to:
optionally operate the piston actuator to bias the piston driver towards the advanced position with a base force such that when the dosage puck is located between the piston driver and the trailing end of the piston, the piston driver exerts a driving force equal to or less than the base force on the trailing end of the piston; and
selectively operate the piston actuator to bias the piston driver towards the advanced position with a pumping force greater than the base force, wherein the pumping force is large enough such that the piston driver moves towards the advanced position and the piston is moved towards the chamber outlet over the stroke distance when the dosage puck is located between the piston driver and the trailing end of the piston.

15. A poultry processing system according to claim 1, wherein, when the piston driver is in the advanced position and the piston is in the rest position, the piston driver does not contact the trailing end of the piston such that a gap is present between the piston driver and the trailing end of the piston.

16. A poultry processing system according to claim 15, wherein the assembly comprises a shim located between the piston driver and the trailing end of the piston, wherein the shim occupies the gap between the piston driver and the trailing end of the piston.

17. A poultry processing system according to claim 1, wherein the piston pump comprises a resting volume defined by a resting distance measured between the forward end of the piston and a terminal stop within the piston chamber, the terminal stop located between the chamber outlet and the forward end when the piston is in the rest position, wherein the selected height of the dosage puck is less than or equal to the resting distance.

18-30. (canceled)

31. A method of processing poultry in a system comprising a system platform and a support ring mounted on a base, the system platform comprising an injection sector comprising a plurality of mounting sites, a bird support mounted on the support ring outside of a perimeter of the system platform, an injection station attached to the system platform at a selected mounting site, the injection station comprising a needle assembly comprising an injection needle and a needle guide, the injection needle configured to move between an injection position and a retracted position along an injection axis, wherein a distal end of the injection needle extends out of a delivery site of the needle guide along the injection axis when the injection needle is in the injection position, and wherein the needle assembly and the needle guide are mounted on the injection carriage, the method comprising:
rotating one or both of the system platform and the support ring relative to each other about a platform axis extending through the system platform, the support ring, and the base, wherein the rotating moves the bird support into and out of alignment with the selected mounting site;
rotating the injection carriage about a carriage axis between a standby position and an actuation position, wherein the injection axis defines a base approach angle relative to the platform axis when the injection carriage is in the actuation position such that the injection needle approaches a bird support positioned at the selected mounting site at the base approach angle; and
positioning a shim between the injection station and the selected mounting site such that, when the injection carriage is in the actuation position, the injection axis approaches the bird support at an adjusted approach angle relative to the platform axis.

32. A method according to claim 31, wherein positioning the shim between the injection station and the selected mounting site rotates the injection axis about the delivery site of the needle guide such that the delivery site of the needle guide is positioned at the same location relative to the platform axis when the injection axis is in the base approach angle and when the injection axis is in the adjusted approach angle.

33. A method according to claim 32, wherein the shim rotates the injection axis about the delivery site 2 degrees or more.

34. A method according to claim 31, wherein the system comprises a piston pump assembly in fluid communication with the injection needle of the injection station, wherein the piston pump assembly comprises: a piston pump comprising a piston movable in a piston chamber of a housing defining a piston chamber, wherein movement of the forward end of the piston along a piston axis within the piston chamber forces fluid in the piston chamber out of the piston chamber through a chamber outlet, and a piston driver configured to be positioned proximate a trailing end of the piston when the piston driver is in the advanced position and the piston is in a rest position, the method comprising:
determining a selected volume of fluid to deliver to the injection needle from the piston pump;
locating a selected dosage puck between the trailing end of the piston and the piston driver to move the piston driver away from the advance position, wherein the selected dosage puck comprises a selected height that corresponds to the selected volume in the pump chamber; and
moving the piston driver to the advance position after locating the selected dosage puck between the trailing end of the piston and the piston driver to move the piston driver away from the advance position, wherein moving the piston driver to the advance position moves the piston towards the chamber outlet over a stroke distance equal to the selected height of the selected dosage puck to move the selected volume of fluid out of the piston chamber through the chamber outlet.

35. A method according to claim 34, wherein, before locating the selected dosage puck between the trailing end of the piston and the piston driver, the method comprises:
determining when a gap is present between the piston driver and the trailing end of the piston when the pump driver is in the advanced position and the piston is in the rest position; and
locating a selected shim between the piston driver and the trailing end of the piston, wherein the selected shim occupies the gap between the piston driver and the trailing end of the piston.

36. (canceled)

37. A method according to claim 34, wherein the piston pump comprises a resting volume defined by a resting distance measured between the forward end of the piston within the piston chamber when the piston is in the rest position, wherein the selected height of the dosage puck is less than or equal to the resting distance.

38-62. (canceled)
